(19) United States
(12) Patent Application Publication
(10) Pub. No.: US 2022/0290149 A1
(43) Pub. Date: Sep. 15, 2022

(54) METHOD OF DETECTION OF FIBRIN CLOTS

(71) Applicant: I-ZUM-X PTY LTD, Hunters Hill (AU)

(72) Inventors: George Bautovich, Hunters Hill (AU); Rakesh Naduvile Veedu, Bull Creek (AU)

(21) Appl. No.: 17/770,241

(22) PCT Filed: Nov. 12, 2020
(86) PCT No.: PCT/AU2020/051228

§ 371 Date: Apr. 19, 2022

(30) Foreign Application Priority Data
Nov. 14, 2019 (AU) 2019904291

(51) Int. Cl. C12N 15/115; C12Q 1/6813; C12N 15/10; C12Q 1/6811

(52) U.S. Cl. CPC C12N 15/115; C12Q 1/6813; C12N 15/1048; C12Q 1/6811; C12N 2310/16; C12N 2310/3231; C12N 2320/13; C12N 2310/3517

(57) ABSTRACT

The use of aptamers for the detection of fibrin and/or blood clots and methods to produce such aptamers.

### TECHNICAL FIELD

The present invention relates to the use of aptamers for the detection of fibrin and/or blood clots and methods to produce aptamers that target fibrin and/or blood clots.

### BACKGROUND ART

Blood clotting is an important process that prevents excessive bleeding when a blood vessel is injured. Fibrin (also called Factor Ia) is a fibrous, insoluble, non-globular protein involved in the clotting of blood. It is formed by the action of the protease thrombin on fibrinogen, which causes the fibrinogen to polymerize. The polymerized fibrin together with platelets forms a haemostatic plug (thrombus or clot) over a wound site. Clots are either stationary (thrombosis) and block blood flow or break loose (embolism) and travel to various parts of the body.

Fibrinogen is a protein present in the blood and is often described as having three nodules held together by a thinner coiled-coiled region. The two end nodules (termed D regions or domains) are alike in consisting of Aα, Bβ and γ chains (COOH ends) while the centre slightly smaller nodule (termed the E region or domain) consists of the same three chains at their amino ends.

Typically, the body will naturally organise the blood clot after the injury into scar tissue. Sometimes, however, clots form on the inside of vessels without an obvious injury. These situations can be dangerous and require accurate diagnosis and appropriate treatment. Whenever there is blood clot in the circulation, there is fibrinolysis. The consequence of fibrinolysis is soluble fibrin degradation products (FDP) starting from the largest size (MW 10 million) down the end product which is D-dimer. FDP are present in the blood of subjects who have clot present in their circulation.

There are two different types of clots:
Venous clots typically form over a period of time; symptoms of venous clots can gradually become more noticeable or may not be noticed at all.
Arterial clots, once formed, cause symptoms immediately. This type of clot prevents oxygen from reaching vital organs. It can cause a variety of complications such as stroke, heart attack, paralysis and intense pain.

The detection of blood clots usually involves venous ultrasound or a CT angiography (CTA). However, it is difficult to detect many clots, particularly venous clots and early clots of either sort (before major symptoms develop).

There is a need to provide more specifically targeted methods for the detection of blood clots; or at least the provision of alternative methods to compliment the previously known methods to treat clot. The present invention therefore seeks to provide an improved or alternative method for the detection of blood clots and targeted therapy of clots.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides an isolated or purified aptamer adapted to bind fibrin. Preferably the fibrin is in a blood clot.

Preferably the aptamer of the present invention does not bind one or more of the following: fibrinogen, fibrin monomer, non-cross-linked fibrin, protofibril, soluble fibrin, fibrin degradation product (FDP), D-dimer or other protein components of plasma. Preferably the aptamer of the present invention does not bind any of the following: fibrinogen, fibrin monomer, non-cross-linked fibrin, protofibril, soluble fibrin, fibrin degradation product (FDP), D-dimer or other protein components of plasma. Preferably the aptamer of the present invention binds fibrin, more preferably insoluble cross-linked fibrin.

Preferably the aptamer of the present invention is labelled with a detection means.

Preferably the aptamer of the present invention is chosen from:

i) the sequences provided in Tables 10 and 11;

ii) any of SEQ ID Nos: 1-21;

iii) SEQ ID NO: 10 or 13;

iv) SEQ ID NO: 16 or 17;

v) sequences which have at least 85% sequence similarity to any one of (i) to (iv); and/or

vi) sequences which have at least 85% sequence identity to any one of (i) to (iv).

The present invention further provides a method for the detection of fibrin in a subject, said method comprising the steps of:

i) administering to the plasma of the subject an aptamer adapted to bind fibrin;

ii) detecting the aptamer bound to the fibrin.

The present invention further provides for the use of purified and isolated aptamers adapted to bind fibrin, for the manufacture of a pharmaceutical composition for the detection of fibrin.

The present invention further provides a kit for the detection of fibrin in the plasma of a subject, said kit comprising:

i) an aptamer adapted to bind fibrin; and

ii) instructions for use.

The present invention further provides a method to produce aptamers that target fibrin using Systematic Evolution of Ligands by EXponential enrichment (SELEX) technology. The method to produce aptamers that target fibrin comprises the steps of:
a) performing SELEX using D-dimer as the positive target protein and a single fibrinogen D domain as the subtraction target protein;
b) performing SELEX using insoluble cross-linked fibrin as the positive target protein and a single fibrinogen D domain as the subtraction target protein; or
c) performing SELEX using peptide fragment 201-216 as the positive target protein.

The above methods may be performed with any of the below in the SELEX medium:

i) no plasma;

ii) plasma from normal people (no clot in their circulation); or

iii) plasma from abnormal patients (clot in their circulation).

The present invention further provides a method to produce aptamers that target fibrin, said method comprising the steps of:
a) performing SELEX using (i) clot as the positive target protein and (ii) plasma from people who do not have clot in their circulation in the SELEX medium; or
b) performing SELEX using (i) clot as the positive target protein and (ii) plasma from patients who have clot abnormally present in their circulation in the SELEX medium.

Preferably the aptamers developed using the above methods target insoluble cross-linked fibrin.

Further aspects of the invention will now be described with reference to the accompanying non-limiting examples and drawings.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a representative flow-chart of EMSA (Electrophoretic Mobility Shift Assay)-SELEX. For the first two selection rounds, TGpr-1 (Target protein; Degradation product of fibrin clot, D-dimer, DD) was incubated with a single-stranded (ss) DNA library of random sequences. As a negative control, rounds 3 to 9 involved the incubation of ssDNA pool with immobilized-SUBpr1, and the unbound ssDNA were incubated with TGpr1. TGpr1-binding ssDNAs were separated from non-binding ssDNA by native gel electrophoresis (5% polyacrylamide gel).

FIG. 2 is a representative flow-chart of AP (Amine binding Plate)-SELEX and MB (Magnetic Bead)-SELEX. A) Immobilized TGpr1 in amine-binding wells were incubated with ssDNA. For enhanced stringency, the wells were washed with aptamer binding buffer prior to extraction of TGpr1-binding ssDNAs with sodium hydroxide treatment. B) Immobilized TGpr1 on tosyl-activated magnetic beads were incubated with ssDNAs. TGpr1-binding ssDNAs were separated from non-binding ssDNAs using a magnetic tube rack, followed by washing with binding buffer to enhance selection stringency. As negative control for both AP-SELEX and MB-SELEX, ssDNAs from rounds three and onwards were incubated with immobilized SUBpr1 (Subtraction protein1; DD minus D protein, the negative control target) on either amine-binding plate or tosyl-activated magnetic beads, and the unbound ssDNAs were collected and incubated with TGpr1 to proceed with the selection.

FIG. 3 is a range of EMSA-SELEX aptamer candidates. A) Unique aptamer candidate sequences that were conserved in round 3 and round 9. B) Enrichment patterns of the aptamer candidates between rounds 3 and round 9. C) Population comparison of EM1 (Electrophoretic mobility Method aptamer candidate 1) in round 3 and round 9. Only EM1 is visible on the pie charts due to the negligible population of EM2-EM6.

FIG. 4 is a range of AP-SELEX aptamer candidates. A) Unique aptamer candidate sequences that were conserved in round 3 and round 10. B) Enrichment patterns of the aptamer candidates between rounds 3 and round 10. C) Population comparison of AP1 (Amine binding Plate method aptamer candidate 1), AP2, and AP3 in round 3. D) Population comparison of AP1, AP2, and AP3 in round 10. E) Population comparison of AP1, AP2, and AP3 combined in round 10.

FIG. 5 is graphs of enzyme linked oligonucleotide assay (ELONA) binding analysis of the three aptamer candidates to TGpr-1 protein.

FIG. 6 is a graph of the relative fluorescence of five aptamers to TGpr-1 protein and SUBpr-1 protein. The test was performed with an ELONA method with fixed aptamer concentration (500 nM) and protein amount (2 ug/well). *p<0.01.

FIG. 7 is a graph of the binding affinity of AP10-01 aptamer to TGpr-1 protein.

FIG. 8 is a graph of the binding affinity of AP10-01-81 aptamer to TGpr-1 protein.

FIG. 9 is a graph showing that AP10-01 aptamer does not display specific binding to blood clot.

FIG. 10 is a graph showing that no significant binding capacity was observed by qPCR quantification at Round 0, 3, 6 and 9

FIG. 11 is photographs of the blood clot targets, displaying stable crosslink structure.

FIG. 12 are images of a denaturing gel analysis and PCR assay showing that the initial RNV31 library is not significantly degraded after 2 h incubation in 75% plasma as demonstrated by gel imaging and PCR assay.

FIG. 13 is a graph of monitoring the selection progress using qPCR method. The enrichment reaches plateau after Round 11, with Round 15 showing similar enrichment with Round 12, 13 and 14.

FIG. 14 shows the sub-libraries of Round 3 (sample 1, 501 nM), Round 9 (sample 2, 509 nM) and Round 15 (sample 3, 595 nM), prepared for next generation sequencing.

FIG. 15 is images of the secondary structure of the identified aptamer candidates.

FIG. 16 is a graph of a representative picture of the relative binding capacity assessment by qPCR.

FIG. 17 is a graph of the PCR efficiency of different aptamer candidates

FIG. 18 is a graph showing aptamer RNV602 displays the strongest binding capacity to blood clot.

FIG. 19 is a graph showing aptamer RNV602 can specifically target blood clot as demonstrated by qPCR based binding assay.

FIG. 20 is a graph showing aptamer RNV605 displayed a medium-high binding affinity to blood clot as demonstrated by qPCR based binding assay.

FIG. 21 is images of aptamers RNV602 and RNV605 display binding to blood clots.

FIG. 22 is a graph showing both aptamers RNV602 and RNV605 display specific binding to blood clots with M11 aptamer as negative control.

FIG. 23 is a graph showing both aptamers RNV602 and RNV605 display specific binding to blood clots with D-dimer and fibrinogen proteins as negative control.

FIG. 24 is a graph showing the 12.5 nM to 800 nM concentration range is not suitable for the measurement of the binding affinity of aptamers RNV605 and RNV602, as demonstrated by fluorescence-based ELONA assay.

FIG. 25 is a graph showing RNV602 and RNV605 display picomolar level binding affinity to blood clots.

FIG. 26 is a graph showing RNV602, RNV605 do not display specific binding to collagen.

FIG. 27 is images showing that aptamer LNA-RNV605 is stable in 90% serum for 6 h.

FIG. 28 is a graph showing LNA modification does not affect the binding capacity of aptamer LNA-RNV605.

FIG. 29 is a schematic of the process of fibrin creation and degradation.

FIG. 30 is images of RNV602 and RNV605 binding to both human and mouse derived blood clots.

FIG. 31 is a graph of the relative binding of RNV744 and RNV745 to fibrinogen-peptide.

FIG. 32 is a graph of RNV746 and RNV747 display binding to fibrinogen-peptide.

### DESCRIPTION OF INVENTION

### Detailed Description of the Invention

The inventors have surprisingly found that aptamers can be used to specifically bind fibrin. The aptamers bind to the target fibrin protein, and a detection means attached to the aptamer allows the presence of the aptamer-clot combination to be detected.

Aptamers are short, single-stranded (ss) DNA or RNA molecules that bind to a specific target. They are typically selected through systematic evolution of ligands by Systematic Evolution of Ligands by EXponential enrichment (SELEX) technology. Aptamers are unique molecules that form three dimensional structures for binding to targets, including proteins. Aptamers do not form heteroduplexes with protein targets; instead, they bind via small structural motifs interacting with particular amino acids on the protein structure.

In contrast to other detection methods, the present method can detect early and/or small clots in plasma, due to the specificity of the fibrin-binding aptamer and the sensitivity of the detection method. The plasma may be from a subject that does not have clots (“normal plasma”) or from a subject that has clots present in their blood (“abnormal plasma”).

### Aptamer

Broadly, according to one aspect of the invention, there is provided a purified and isolated aptamer for binding to the fibrin protein. This aptamer allows the detection of fibrin in a subject, for example fibrin in a blood clot in a subject.

The term “fibrin” refers to one or more elements from the following list: protofibril, non-cross-linked fibrin and insoluble cross-linked fibrin. More preferably, the term “fibrin” refers to insoluble cross-linked fibrin.

Preferably, the SELEX iterations should be carried out using insoluble cross-linked fibrin as the target and plasma from subjects with clot in the SELEX medium. Aptamers which bind the fibrin attach to the insoluble cross-linked fibrin and aptamers which bind the plasma constituents are soluble in the medium. The insoluble cross-linked fibrin can then be separated from the soluble medium. This results in a method to produce aptamers that preferentially target the insoluble cross-linked fibrin which is the main constituent of clot (thrombus) and which do not bind soluble other elements in the blood of subjects who have clots present in their circulation, including: fibrinogen, fibrin monomer, non-cross-linked fibrin, protofibril, soluble fibrin, fibrin degradation product (FDP), and D-dimer.

For example, in one aspect of the invention, there is provided an aptamer which specifically targets to a region of the fibrin protein. Preferably, the location targeted by the aptamer is present in fibrin, but not present or accessible in the fibrin precursor molecule—fibrinogen, the intermediate fibrinogen degradation products—FDP, or the end degradation product of normal fibrin metabolism—D-dimer. Preferably, the aptamer does not bind to any other protein components of plasma in the blood of subjects who have clot abnormally present in their blood.

For example, in one aspect of the invention, there is provided an aptamer which specifically targets a region of the fibrin protein. Preferably, the location targeted by the aptamer is present in fibrin, but not present or accessible in the fibrin precursor molecule—fibrinogen, the intermediate fibrin degradation products—FDP, or the end degradation product of normal fibrin metabolism—D-dimer. Preferably, the aptamer does not bind to any other protein components of plasma in the blood of subjects who have clot present in their blood. Preferably the aptamer of the present invention does not bind one or more of the following: fibrinogen, fibrin monomer, non-cross-linked fibrin, protofibril, soluble fibrin, fibrin degradation product (FDP), D-dimer or other protein components of plasma. Preferably the aptamer of the present invention does not bind any of the following: fibrinogen, fibrin monomer, non-cross-linked fibrin, protofibril, soluble fibrin, fibrin degradation product (FDP), D-dimer or other protein components of plasma.

Preferably the aptamer has a binding affinity (dissociation constant Kd) for insoluble cross-linked fibrin of between 30 pM and 100 nM, more preferably between 100 pM and 450 pM when tested by qPCR-based binding capacity assay or ELONA assay.

Preferably the aptamer of the present invention has a binding affinity (dissociation constant Kd) for fibrinogen, fibrin monomer, non-cross-linked fibrin, protofibril, soluble fibrin, fibrin degradation product (FDP), D-dimer or other protein components of plasma of greater than 1000 nM when tested by qPCR-based binding capacity assay or ELONA assay.

The plasma may be from a subject that does not have clots (“normal plasma”) or from a subject that has clots present in their blood (“abnormal plasma”). Plasma from subjects who have clot present in their circulation have fibrin degradation products (FDP) of many sizes starting from about 10 million MW down to D-dimer. The end product of fibrinolysis of cross-linked insoluble fibrin is D-dimer.

The present detection test may be carried out on the plasma or blood of subjects with no symptoms of clot, with suspected symptoms of clot or with known clot. For example, the present detection method may be used during treatment for clot to determine if or when the clot has been degraded by the treatment. The detection test can be carried out in vivo, in vitro or in vitro.

By in vitro, it is meant that blood may be removed from a subject and combined with the aptamer to detect the presence of fibrin. By in vivo, it is meant that the aptamer may be administered to a subject and the presence of aptamer-bound fibrin within the body detected by a screening or imaging method. By in vitro, it is meant that the aptamer is administered to a subject, then the blood of the subject is withdrawn and the presence of aptamer-bound fibrin in the blood sample detected. For example, the relative amount of aptamer administered per blood volume of the subject may be calculated before administration and then the amount of free aptamer in the withdrawn blood determined after administration, with the difference representing the amount of aptamer-bound fibrin within the body.

Preferably the aptamer is chosen from the sequences provided in Tables 10 and 11; that is, any one or more of SEQ ID Nos: 1-21 and combinations or cocktails thereof. More preferably, the aptamer is SEQ ID NO: 10 or 13 (RNV602 or RNV605) or SEQ ID NO: 16 or 17 (RNV744 or RNV745). This includes sequences which can hybridise to such sequences under stringent hybridisation conditions, sequences complementary thereto, sequences containing modified bases, modified backbones, and functional truncations or extensions thereof which bind to the fibrin protein. In certain embodiments, the original aptamers may include mismatches or multiple chemical modifications, e.g., to accommodate variants, for subsequent detection. Hence, certain aptamers may have about or at least about 15% sequence mutation, e.g., 15%, 14%, 13%, 12%, 11%, 10%, 9%, 8%, 7%, 6%, 5%, 4%, 3%, 2%, or 1%. The aptamers of the present invention may have at least 85%, 86%, 87%, 88%, 89%, 90%, 91%, 92%, 93%, 94%, 95%, 96%, 97%, 98% or 99% sequence identity or may have at least 85%, 86%, 87%, 88%, 89%, 90%, 91%, 92%, 93%, 94%, 95%, 96%, 97%, 98% or 99% sequence similarity to the aptamers of Tables 10 and 11; that is, any one or more of SEQ ID NOs: 1-21.

The invention extends also to a combination of two or more purified and isolated aptamers capable of binding to a selected target protein, including a construct comprising two or more such aptamers.

The invention extends, according to a still further aspect thereof, to cDNA or cloned copies of the aptamers of the invention, as well as to vectors containing the aptamers of the invention. The invention extends further also to cells containing such sequences and/or vectors.

To increase the half-life, stability and/or binding affinity of aptamers, particularly nucleic acid aptamers, the selected aptamers are chemically modified, for example, by replacing the 2′ position with either a fluoro- (F), amino- (NH2), or O-methyl (OCH3) group, and by capping the 3′ end with inverted thymidine to increase nuclease resistance while also enhancing binding affinity. The aptamers can be modified in-SELEX and/or post-SELEX.

For in-SELEX modifications, aptamers with the desired modifications are directly isolated from a DNA or RNA library containing modified nucleotides that are compatible with DNA or RNA polymerases. The aptamers that can be modified by this method include 2′-am inopyrimidines, 2′-fluoropyrimidines, 2′-O-methyl nucleotides and locked nucleic acids (LNA).

As for post-SELEX modifications, different modifications at various positions (e.g., base, 2′-position, sugar ring, phosphate group) can be introduced to selected aptamers during solid-phase chemical synthesis. Since the affinity/specificity and function of an aptamer depend on its structure, post-SELEX modification may affect the inherent properties and folding structures of the original aptamers, thereby compromising the binding affinity. Therefore, it is necessary to precisely tailor modifications to optimize the desired functions.

Various chemical modifications to stabilize aptamers are disclosed in the art, for example, Gijs M., et al., Nuclear Medicine and Biology, 43(4): 253-271 (2016); and Yu Y, et al. International Journal of Molecular Sciences, 17(3): 358 (2016).

Preferably the aptamer is modified to increase stability and reduce the rate of in vivo degradation. For example, the aptamer may be modified by the inclusion of one or more of the following modifications: locked nucleic acid-nucleotides (LNA-nucleotides), 2′-Fluoro nucleotides, 2′-O-Methyl nucleotides, phosphorodiamidate morpholino nucleotides, unlocked nucleic acid nucleotides, L-DNA/L-RNA nucleotides and inverted-dT nucleotides.

Preferably the aptamer is labelled with a detection means. Examples of detection means include: radiotracers, fluorescent dyes (FAM, Cy5, Dy647 and others), drug molecules, electrochemical signalling molecules, magnetic and polymeric nanoparticles, lipids and liposomes, magnetic labels (for example for Nuclear Magnetic Resonance (NMR) imaging), iodine X-ray blocking compounds, radiotracers (for example for Positron Emission Tomography (PET) [especially Gallium-68]), Technetium-99m for Single Photon Emission Computerised Tomography (SPECT). Other suitable labelling means, particularly radioactive tracers, may be used in the present invention, according to the knowledge, skills and facilities available to the skilled person.

By “isolated” or “purified” is meant material that is substantially or essentially free from components that normally accompany it in its native state. For example, an “isolated aptamer” or “purified aptamer”, as used herein, may refer to an aptamer that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject. In the context of oligonucleotides or protein, “isolating” refers to the recovery of oligonucleotides or protein from a source, e.g., cells.

An aptamer can be said to be “directed to” or “targeted against” a target protein sequence with which it binds.

In certain embodiments, the aptamer has sufficient sequence complementarity to a target protein (i.e., the protein to which it is adapted to bind) to bind in an effective manner.

Selected aptamers can be made shorter, e.g., about 10 bases, or longer, e.g., about 100 bases, and include a small number of mismatches, as long as the sequence is sufficiently complementary to the target protein to bind.

The aptamer lengths should be sufficient to the target protein sequence to form a stable aptamer-protein complex. The length of the aptamer with the target protein sequence may be as short as 8-11 bases, but can be 20-80 bases or more, e.g., up to 100 bases, including all integers in between these ranges. An aptamer of about 50-80 bases is generally long enough to have a unique complementary sequence. In certain embodiments, a minimum length of complementary bases may be required to achieve the requisite binding affinity.

In certain embodiments, aptamers with the original sequence may be used, as long as a complex is formed between the aptamer and target protein sequence is sufficiently stable for subsequent detection. Hence, certain aptamers may have about or at least about 15% sequence mutation, e.g., 15%, 14%, 13%, 12%, 11%, 10%, 9%, 8%, 7%, 6%, 5%, 4%, 3%, 2%, 1%. The number of mismatches allowed will depend on the length and tertiary structure of the aptamer. Although such an aptamer is not necessarily identical with the original sequence, it is effective to stably and specifically bind to the target sequence, such that detection of the target protein is possible.

Additional examples of variants include aptamers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of the sequences provided in Tables 10 and 11; that is, any one or more of SEQ ID Nos: 1-21 or more preferably, SEQ ID NO: 10 or 13 or SEQ ID NO: 16 or 17.

The aptamer sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the aptamer, the greater the homology required to obtain selective binding to the target protein. Consequently, where an aptamer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the aptamers set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The aptamers of the present invention may have regions different from the original aptamer sequences. It is not necessary for an aptamer to have exact the identical sequence with the original sequences. For example, the aptamers may have continuous stretches of at least 4 or 5 bases that are identical to the original sequence, preferably continuous stretches of at least 6 or 7 bases that are identical to the original sequence, more preferably continuous stretches of at least 8 or 9 bases that are identical to the original sequence. The remaining stretches of aptamers sequence may be intermittently identical with the original sequence; for example, the remaining sequence may have an identical base, followed by a non-identical base, followed by an identical base. Alternatively (or as well) the aptamers sequence may have several stretches of identical sequence (for example 3, 4, 5 or 6 bases) interspersed with stretches of less than perfect region. Such sequence mismatches will preferably have no or very little loss of target protein binding activity.

Preferably the aptamer is 10 to 100 nucleotides in length.

The aptamers used in accordance with this invention may be conveniently made through the well-known technique of solid phase synthesis. Equipment for such synthesis is sold by several vendors including, for example, Applied Biosystems (Foster City, Calif.). One method for synthesising aptamers on a modified solid support is described in U.S. Pat. No. 4,458,066.

Any other means for such synthesis known in the art may additionally or alternatively be employed. It is well known to use similar techniques to prepare aptamers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The aptamers of the invention are synthesised in vitro and do not include aptamers of biological origin, or genetic vector constructs designed to direct the in vivo synthesis of aptamers. The aptamers of the invention may also be mixed, encapsulated, conjugated or otherwise associated with other molecules, molecule structures or mixtures of compounds, as for example, liposomes, receptor targeted molecules etc.

The aptamers may be formulated for oral, topical, parenteral or other delivery, particularly formulations for injectable delivery. The formulations may be formulated for assisting in uptake, distribution and/or absorption at the site of delivery or activity. Preferably the aptamers of the present invention are formulated for delivered via injection.

### Method of Detection

According to a further aspect of the invention, there is provided a method for the detection of fibrin in a subject, said method comprising the steps of:
a) administering to the plasma of the subject a purified and isolated aptamer adapted to bind fibrin;
b) detecting the aptamer bound to the fibrin.

The plasma of the subject may still be in the subject (i.e. the aptamer is injected or otherwise introduced into the subject) or the plasma may be withdrawn from the subject before testing. If the aptamer is introduced into the subject, a sample of the plasma containing the aptamer may be removed from the subject for testing, or the subject may be exposed to a detection means that detects the in situ in vivo presence of the aptamer at the site of clot (e.g. a PET scan or X-ray).

Preferably the aptamer of the present invention does not bind one or more of the following: fibrinogen, fibrin monomer, non-cross-linked fibrin, protofibril, soluble fibrin, fibrin degradation product (FDP), D-dimer or other protein components of plasma. Preferably the aptamer of the present invention does not bind any of the following: fibrinogen, fibrin monomer, non-cross-linked fibrin, protofibril, soluble fibrin, fibrin degradation product (FDP), D-dimer or other protein components of plasma. Preferably the aptamer of the present invention binds fibrin, more preferably insoluble cross-linked fibrin.

The aptamer may be labelled with a detection means. Examples of detection means include: radiotracers, fluorescent dyes (FAM, Cy5, Dy647 and others), drug molecules, electrochemical signalling molecules, magnetic and polymeric nanoparticles, lipids and liposomes, magnetic labels (for example for Nuclear Magnetic Resonance (NMR) imaging), iodine X-ray blocking compounds, radiotracers (for example for Positron Emission Tomography (PET) [especially Gallium-68]), Technetium-99m for Single Photon Emission Computerised Tomography (SPECT).

Preferably the aptamer of the present invention is chosen from:

i) the sequences provided in Tables 10 and 11;

ii) any of SEQ ID Nos: 1-21;

iii) SEQ ID NO: 10 or 13;

iv) SEQ ID NO: 16 or 17;

v) sequences which have at least 85% sequence similarity to any one of (i) to (iv); and/or

vi) sequences which have at least 85% sequence identity to any one of (i) to (iv).

The subject with the fibrin and/or blood clot may be a mammal, including a human.

The blood clot may be a venous clot or arterial clot. The clot or fibrin may be attached to a circulatory vessel wall, may be free moving in the circulatory system, or may be outside the circulation in the tissues of the patient.

Detection of fibrin and/or blood clot by the methods of the present invention may be followed by treatment for the clot.

### Compositions

There is also provided a pharmaceutical composition for the detection of fibrin in a subject, the composition comprising:

a) a purified and isolated aptamer adapted to bind fibrin as described herein; and

b) one or more pharmaceutically acceptable carriers and/or diluents.

The composition may comprise about 1 nM to 1000 nM of each of the desired aptamers of the invention. Preferably, the composition may comprise about 10 nM to 500 nM, most preferably between 1 nM and 10 nM of each of the aptamers of the invention.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Remington: The Science and Practice of Pharmacy, 22nd Ed., Pharmaceutical Press, PA (2013).

In a more specific form of the invention there are provided pharmaceutical compositions comprising therapeutically effective amounts of one or more aptamers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hyaluronic acid may also be used. Such compositions may influence the physical state, stability, rate of in vivo release, and rate of in vivo clearance of the present aptamers. See, for example, Remington: The Science and Practice of Pharmacy, 22nd Ed., Pharmaceutical Press, PA (2013). The compositions may be prepared in liquid form, or may be in dried powder, such as a lyophilised form.

It will be appreciated that pharmaceutical compositions provided according to the present invention may be administered by any means known in the art. The pharmaceutical compositions for administration are administered by injection, orally, topically or by the pulmonary or nasal route. For example, the aptamers may be delivered by intravenous, intra-arterial, intraperitoneal, intramuscular or subcutaneous routes of administration. The appropriate route may be determined by one of skill in the art, as appropriate to the condition of the subject under treatment. Preferably, the aptamers are parenterally delivered, for example via injection for example intravenous, subcutaneous or intramuscular administration.

The delivery of a therapeutically useful amount of aptamers may be achieved by methods previously published. For example, delivery of the aptamer may be via a composition comprising an admixture of the aptamer and an effective amount of a block copolymer. An example of this method is described in US patent application US20040248833.

It may be desirable to deliver the aptamer in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro and in vivo delivery methods. It has been shown that large unilamellar vesicles can encapsulate a substantial percentage of an aqueous buffer containing large macromolecules. RNA and DNA can be encapsulated within the aqueous interior and be delivered in a biologically active form (Fraley, et al., Trends Biochem. Sci. 6:77, 1981).

The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The aptamer may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular subject and condition.

The aptamers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention; i.e. salts that retain the desired biological activity of the parent compound and do not impart undesired toxicological effects thereto. For aptamers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be via topical (including ophthalmic and mucous membranes, as well as rectal delivery), pulmonary (e.g., by inhalation or insufflation of powders or aerosols, including by nebulizer, intratracheal, intranasal, epidermal and transdermal), oral or parenteral routes. Parenteral administration includes intravenous, intra-arterial, subcutaneous, intraperitoneal, intraocular or intramuscular injection or infusion; or intracranial, e.g., intrathecal or intraventricular, or lumbar puncture, administration. Aptamers with at least one 2′-O-methoxyethyl modification are believed to be particularly useful for parenteral administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

Preferably, the sequence of the present invention is delivered via a parenteral route. For example, the aptamer may be administered via intravenous, intramuscular or subcutaneous injection for diagnosis or for systemic treatments. More preferably, the aptamers of the invention are delivered via intravenous injection.

There is also provided the use of purified and isolated aptamers as described herein, for the manufacture of a pharmaceutical composition for the detection of fibrin. This aptamer allows the detection of fibrin in a subject, for example fibrin in a blood clot in a subject.

There is also provided a kit for the detection of fibrin in the plasma of a subject, said kit comprising:

a) a purified and isolated aptamer adapted to bind fibrin; and

b) instructions for use.

The contents of the kit can be lyophilized, and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the aptamers may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

In an embodiment, the kit of the present invention comprises a composition comprising a therapeutically effective amount of a purified and isolated aptamer adapted to bind fibrin as described herein. In an alternative embodiment, the formulation is in pre-measured, pre-mixed and/or pre-packaged. Preferably, the kit is for parenteral administration and the solution is sterile.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for administering the formulations of the present invention. In one embodiment, the instructions indicate that the formulation of the present invention is suitable for detection of fibrin and/or blood clots. Such instructions may also include instructions on dosage, as well as instructions for administration.

The aptamers and suitable excipients can be packaged individually so to allow a practitioner or user to formulate the components into a pharmaceutically acceptable composition as needed. Alternatively, the aptamers and suitable excipients can be packaged together, thereby requiring de minimus formulation by the practitioner or user. In any event, the packaging should maintain chemical, physical, and aesthetic integrity of the active ingredients.

### Method for Aptamer Development

According to a further aspect of the invention, there is provided a method to produce aptamers that target fibrin, said method comprising the steps of:
a) performing SELEX using D-dimer as the positive target protein and a single fibrinogen D domain as the subtraction target protein.

If the D-dimer is used as the positive target protein and a single fibrinogen D domain as the subtraction target protein, the aptamers remaining are those that target the cross-link present in the D-dimer but not in the single fibrinogen D domain.

According to a further aspect of the invention, there is provided a method to produce aptamers that target fibrin, said method comprising the steps of:
a) performing SELEX using insoluble cross-linked fibrin as the positive target protein and a single fibrinogen D domain as the subtraction target protein.

If insoluble cross-linked fibrin is used as the positive target protein and a single fibrinogen D domain as the subtraction target protein, the aptamers remaining are those that target the cross-link present in the insoluble cross-linked fibrin but not in the single fibrinogen D domain.

According to a further aspect of the invention, there is provided a method to produce aptamers that target fibrin, said method comprising the steps of:
a) performing SELEX using peptide fragment 201-216 as the positive target protein.

Peptide fragment 201-216 is fibrinogen peptide Cys-Asn-Ile-Pro-Val-Val-Ser-Gly-Lys-Glu-Cys-Glu-Glu-Ile-Ile-Arg (Sci Rep. 2013; 3: 2604, doi: 10.1038/srep02604) in the Bβ chain of fibrinogen. It forms an uncovered region that develops in the fibrin clot during clot formation.

The SELEX of the above methods can be carried out using plasma in the SELEX buffer, or with no plasma in the SELEX buffer. If plasma is used, the plasma may be from a subject that does not have clot (normal plasma) or may be from a subject that does have clot in the circulation (abnormal plasma). A benefit of using plasma in the buffer during the SELEX process is that the aptamers produced are those that are more stable in plasma and therefore can be administered into blood. Only those aptamers that are stable in plasma will be stable enough to be part of the SELEX selection process. If an aptamer is unstable in plasma, that aptamer is not stable enough to go through SELEX in a buffer containing plasma.

The present invention further provides a method to produce aptamers that target fibrin, said method comprising the steps of:
a) performing SELEX using (i) clot as the positive target protein and (ii) plasma from patients who have clot abnormally present in their circulation in the SELEX medium.

The present invention further provides a method to produce aptamers that target fibrin, said method comprising the steps of:
a) performing SELEX using (i) clot as the positive target protein and (ii) plasma from patients who have clot abnormally present in their circulation in the SELEX medium.

Preferably, the above methods use plasma which is obtained from a different subject for each round of SELEX, and the last round of SELEX uses plasma pooled from all of the previous rounds of SELEX.

Preferably the aptamers developed using the above methods target insoluble cross-linked fibrin.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. Size, displacement and field strength etc.). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Aptamer Development

The process termed “systematic evolution of ligands by exponential enrichment” (SELEX) was used for aptamer development against the target protein (TGpr1-D-dimer). In SELEX, aptamers are developed by iterative rounds of target incubation with a single-strand nucleotide library, extraction of target-binding nucleotides and their amplification. The process is typically repeated 8-12 rounds to enrich for high-affinity aptamers.

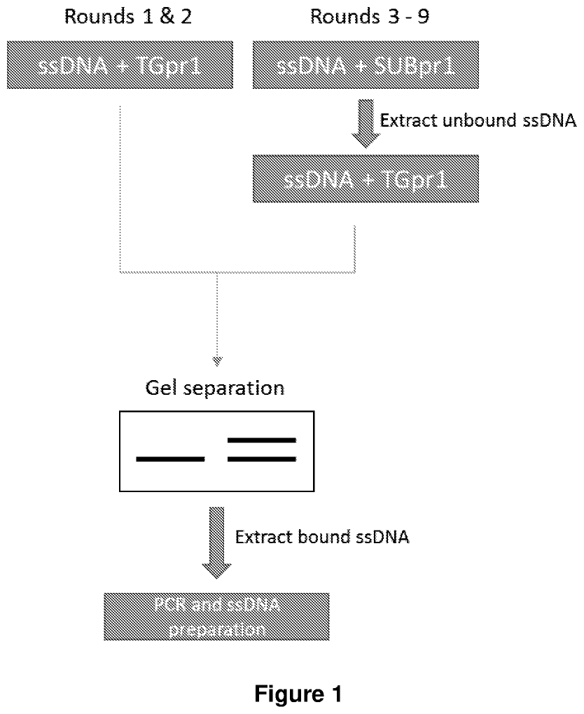

Three separate SELEX experiments were conducted. The first SELEX experiment involved the incubation of ssDNA libraries with the TGpr1 protein in free solution, the TGpr1-binding ssDNA was then separated from non-binding ssDNA by native gel electrophoresis (EMSA-SELEX). As a negative control, enriched pools of ssDNA were incubated with a negative-control protein (SUBpr1), which were immobilized on tosyl-activated magnetic beads, prior to incubation with TGpr1 (FIG. 1). Nine rounds of selection were completed, which generated a sub-library of ssDNAs. This was sequenced and analysed to detect conserved and enriched DNA sequences that bind to TGpr1.

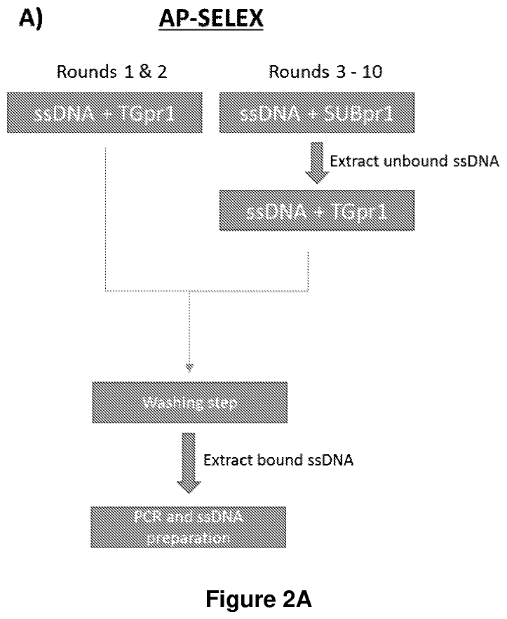

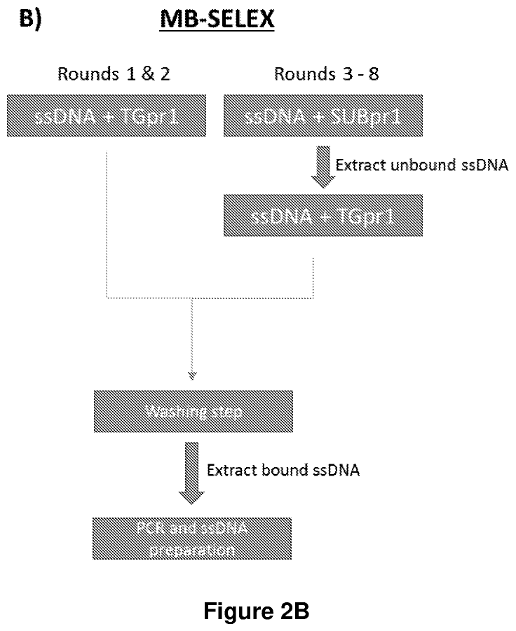

Two additional SELEX experiments were carried out by immobilizing TGpr1 on (i) amine binding plates (AP-SELEX) and (ii) tosyl-activated magnetic beads (MB-SELEX) (FIG. 2). In both methods, the rounds started with 2000 pmol of ssDNA library with 20 pg/well of TGpr1 for AP-SELEX and 20 μg/100 μL of TGpr1 (μg)/dynabeads (μL) for MB-SELEX. Afterwards, all selection rounds were carried out with 100 pmol of enriched ssDNA (Table 1). To eliminate non-specific binding, the subtraction protein (SUBpr1) was immobilised on both the amine-binding plates and the tosyl-activated magnetic beads to eliminate non-specific binding aptamer by firstly incubating the ssDNA with the immobilized SUBpr1 and transferring the unbound ssDNA to the immobilized TGpr1 (FIG. 2). For increased selection stringency, unbound and loosely bound ssDNAs were washed away with the aptamer binding buffer with increasing volume as the selection progressed (Table 1). Ten rounds of selection for AP-SELEX and eight rounds of selection for MB-SELEX were completed.

TABLE 1
Progress summary of AP-SELEX and MB-SELEX.

| | AP-SELEX | | MB-SELEX | |
|---|---|---|---|---|
| SELEX | ssDNA | Washing | ssDNA | Washing |
| Rounds | (pmol) | volume (mL) | (pmol) | volume (mL) |
| 1 | 2000 | 0.2 | 2000 | 0.5 |
| 2 | 100 | 0.6 | 100 | 2 |
| 3 | 100 | 3 | 100 | 3 |
| 4 | 100 | 4 | 100 | 4 |
| 5 | 100 | 5 | 100 | 6 |
| 6 | 100 | 7 | 100 | 7 |
| 7 | 100 | 8 | 100 | 8 |
| 8 | 100 | 9 | NA | NA |
| 9 | 100 | 10 | NA | NA |
| 10 | 100 | 11 | NA | NA |

### SELEX Products (Enriched Aptamer) Sequences

Samples from EMSA-SELEX, AP-SELEX, and MB-SELEX were prepared for sequencing to identify aptamer candidates. To do so, dsDNA of the enriched pools were prepared for all the SELEX experiments and sent for next generation sequencing. Samples were desalted with AMPure and sequenced with Illumina MiSeq.

The third rounds (with the exception of MB-SELEX) and the final rounds of each SELEX experiment (Table 2) were sequenced, then screened for conserved and enriched sequences to identify strong aptamer candidates.

TABLE 2
Details of enriched SELEX products for next generation sequencing.

| Sample name/round # | Sample ID | A280 | 260/280 | 260/230 | ng/uL |
|---|---|---|---|---|---|
| EMSA-SELEX/03 | EM1 | 2.556 | 1.87 | 1.46 | 127.8 |
| EMSA-SELEX/09 | EM2 | 1.214 | 1.91 | 1.16 | 60.7 |
| AP-SELEX/03 | AP1 | 0.899 | 1.81 | 1.02 | 45 |
| AP-SELEX/10 | AP2 | 0.726 | 1.82 | 0.56 | 36.3 |
| MB-SELEX/08 | MB1 | 0.8 | 1.86 | 0.86 | 26.4 |

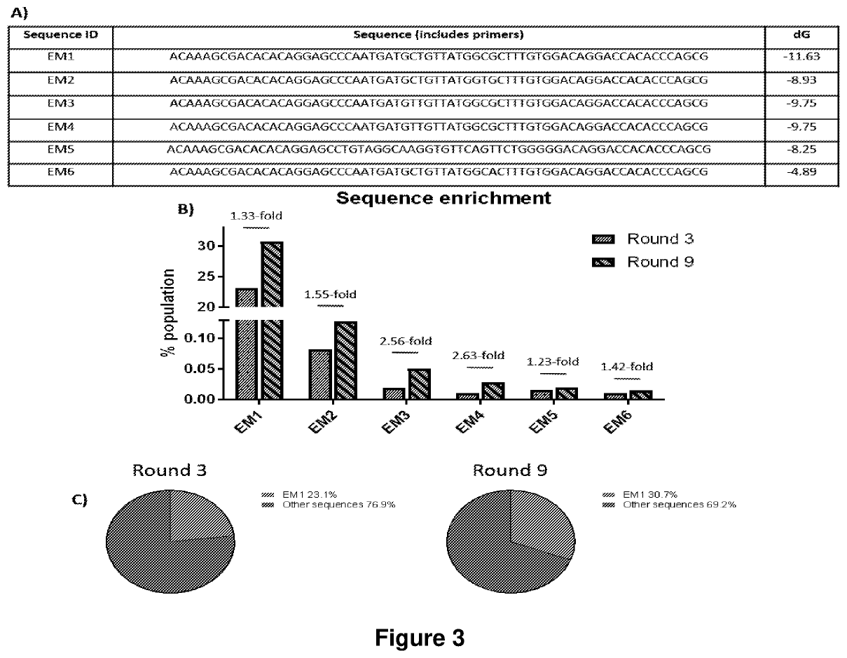

The sequences of EMSA-SELEX were collected, and six unique sequences were identified in round 3 that were conserved and enriched in round 10 (FIG. 3A). As shown in FIG. 3A, EM1 enriched in round 3 to round 9 by 1.33-fold and maintained the highest population in both rounds. Samples EM2-EM6 also showed enrichment from round 3 to round 9 but had negligible populations in both rounds (FIG. 3B, C). The ability of EM1 to maintain its high population throughout EMSA-SELEX suggests that it would make a strong aptamer candidate to test in binding assays with TGpr1.

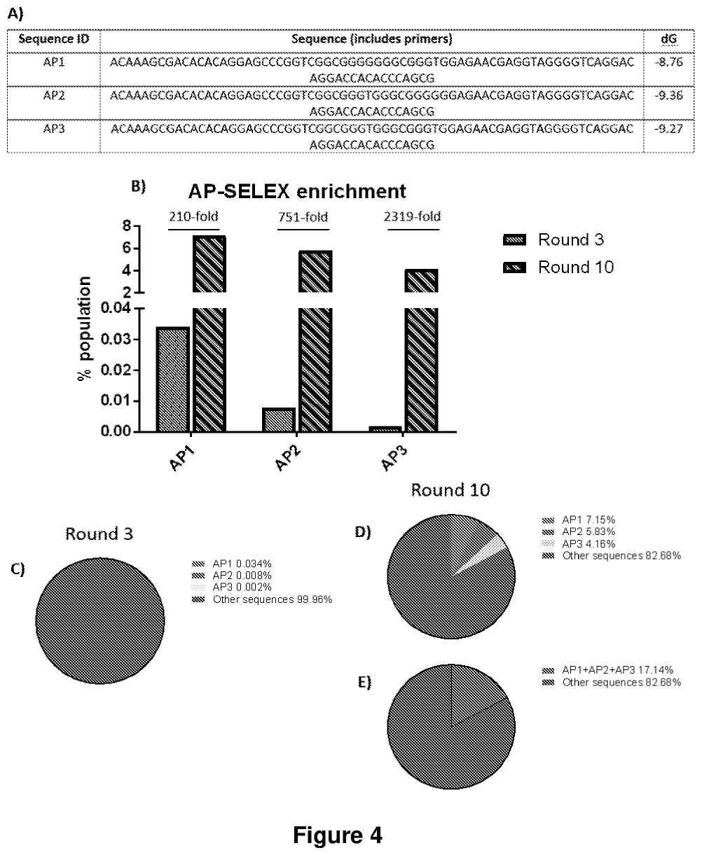

Sequence data from AP-SELEX rounds 3 and 10 were collected and three unique sequences were identified that were fully conserved in both rounds (FIG. 4A). From round three to round ten of selection, AP1 was shown to be enriched by 210-fold, AP2 by 751-fold, and AP3 by 2319-fold (FIG. 4B). These results suggest that the three sequences were enriched because of repetitive binding with TGpr1 and amplification throughout AP-SELEX. Furthermore, each of the AP1, AP2, and AP3 has increased its population within the ssDNA library (FIG. 4C, D). Nearly all of the round ten products maintained identical sequence motifs. Indeed, as seen on FIG. 4A, all three AP samples are 95% similar and collectively enriched by 393-fold (FIG. 4E). Therefore, the three AP samples are considered as strong candidates for TGpr1-specific aptamers.

For MB-SELEX, round 8 products were sequenced and 54 unique sequences were collected. Interestingly, three of the highest enriched sequences matched with AP1, AP2, and AP3. This finding indicates that both AP-SELEX and MB-SELEX led to the enrichment of similar sequences, which further suggests that AP1, AP2, and AP3 are strong aptamer candidates.

### Best Aptamer Candidates for Binding Analysis

After careful analysis of all the sequences obtained, synthesis of aptamer candidates EM1, AP1 and AP3 (Table 3) for preliminary binding assay experiments for their sequence conservation, enrichment, and stability (low dG values) was carried out. EM1 was chosen because it maintained a high population percentage from round 3 to round 9 with moderate enrichment (FIG. 3B, C). AP1 and AP3 were chosen for their significant enrichment from round 3 to round 10, which led to a significant enrichment by up to 2319-fold (FIG. 4B-E). In addition, both AP1 and AP3 show 97.5% similarity.

TABLE 3
Aptamer candidates sent for synthesis.

| SEQ | | | |
|---|---|---|---|
| ID NO | Seq Name | Sequence (includes primers) | dG |
| 1 | EM1 | 5′ Biotin- | -11.63 |
| | | ACAAAGCGACACACAGGAGCCCAATGATGCTGTTATGG | |
| | | CGCTTTGTGGACAGGACCACACCCAGCG-3′ | |
| 2 | AP1 | 5′ Biotin- | -8.76 |
| | | ACAAAGCGACACACAGGAGCCCGGTCGGCGGGGGGGC | |
| | | GGGTGGAGAACGAGGTAGGGGTCAGGACAGGACCACA | |
| | | CCCAGCG-3′ | |
| 3 | AP3 | 5′ Biotin- | -9.27 |
| | | ACAAAGCGACACACAGGAGCCCGGTCGGCGGGTGGGC | |
| | | GGGTGGAGAACGAGGTAGGGGTCAGGACAGGACCACA | |
| | | CCCAGCG-3′ | |

### Example 2

### Validation of Aptamers

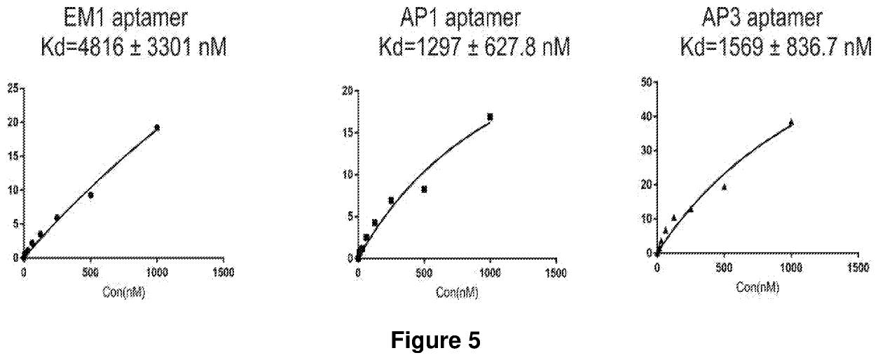

Experiments were undertaken to verify the binding affinity of the three aptamers using a highly sensitive binding assay called ELONA (Enzyme Linked Oligonucleotide Assay, see FIG. 5 for method illustration) to confirm the binding and to determine an accurate K_{d} values. Biotin label were added to the synthesized sequences to facilitate binding analysis by ELONA.

None of the selected aptamers showed high binding affinity as the K_{d} values were above 1000 nM (FIG. 5) which will be non-specific.

The next generation sequencing data of the selected aptamer pool was revisited, and five different candidates without the primer binding regions as listed in Table 4 were selected.

TABLE 4
Newly identified aptamers after bioinformatics analysis against
TGpr-1.

| SEQ | | | |
|---|---|---|---|
| ID NO | Seq Name | New Aptamer sequences after bioinformatic study | Energy |
| 4 | EM9-02 | AAGGGAAAGTGTATACACTATGGCT | -3.2 |
| 5 | MB8-01 | CGGTCGGCCGGGGGGCGGGTGGAGAACGAGGTAGGGGTCA | -4.2 |
| 6 | AP10-01 | ACAGTGTGGGCGGGCGGGTGGTTTGGAGCTGGTAGCGGTA | -1.4 |
| 7 | AP10-06 | TCATCGTGGGTGGGTTGGGTGGAACGACGTAGAGGGGGTG | -2.26 |
| 8 | AP10-07 | CGTTCGGCGGGGGGGCGGGTGGAGAACGAGGTAGGGGTCA | -4.4 |

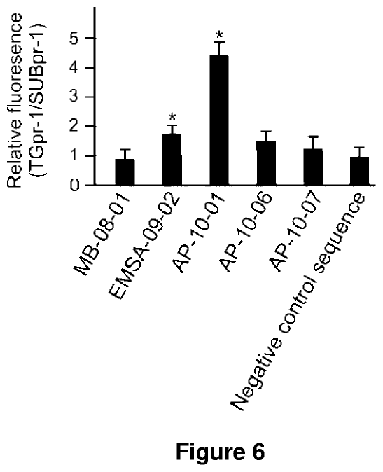

According to initial screening tests as demonstrated in FIG. 6, the binding ratio of the five aptamers to TGpr-1 and SUBpr-1 proteins show different patterns. While AP10-01 and EM09-02 aptamers display higher binding ratio (4.21 and 1.85 respectively) compared with negative control aptamer, aptamers MB08-01 (0.81), AP10-06 (1.31), AP10-07 (1.23) did not display significant difference. This test demonstrates that AP10-01 is a better candidate and was therefore selected for further binding analysis.

### Binding Analysis of AP10-01

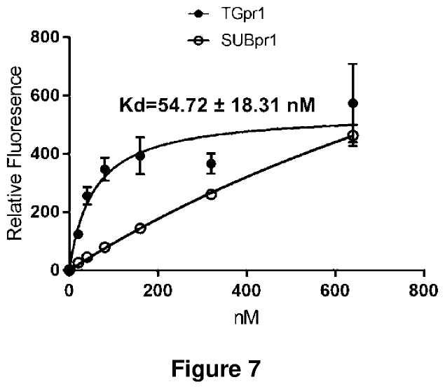

The binding affinity of AP10-01 aptamer to TGpr-1 protein and to the negative control protein SUBpr-1 was analysed by ELONA assay. As demonstrated by FIG. 7, this aptamer displays excellent binding to TGpr1 with a K_{d} value of 54 nM. On the other hand, the binding of AP10-01 aptamer to the negative control SUBpr1 protein shows no binding pattern. This experiment was repeated multiple times, and the results are very reproducible.

### Increasing the Size of AP10-01 Aptamer:

The total size of aptamer AP10-01 is 40 nucleotides. Primer flanking region were attached in order to increase the pharmacokinetic properties in vivo. The resulting aptamer named ‘AP10-01-81’ (Table 5) was synthesized using our oligonucleotide synthesizer in 1 micromole scale using standard phosphoramidite chemistry.

TABLE 5
Sequence details of AP10-01-81 aptamer

| SEQ ID | | | |
|---|---|---|---|
| NO | Seq Name | Length | Aptamer sequence |
| 9 | AP_10_01_81 | 81 | GGACAGGACCACACCCAGCGACAGTGTGGGCGGGC |
| | | | GGGTGGTTTGGAGCTGGTAGCGGTAGGCTCCTGTGT |
| | | | GTCGCTTTGT |

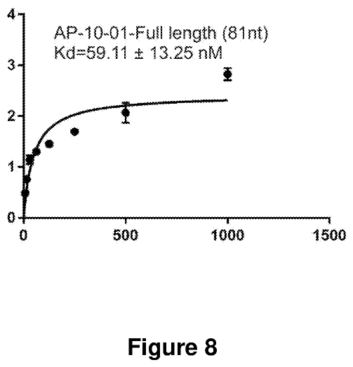

Binding analysis of AP10-01-81: ELONA analysis revealed that the full-length AP-10-01 aptamer is identical to the AP10-01 aptamer in binding TGpr-1 with a K_{d} value of 59 Nm (FIG. 8). This shows that the truncated 40 nucleotide long aptamer may be a better candidate for in vitro analysis. However, the longer variant could serve a better molecule for in vivo applications. Again, AP10-01-81 did not show any binding to the negative protein, SUBpr-1.

Improving the Stability of AP10-01 & AP10-01-81 Aptamers with LNA-Nucleotides and an Inverted-dT Nucleotides

To further improve the stability of AP-10-01 aptamer and potentially improve the binding property of it, AP10-01 & AP10-01-81 aptamers with LNA-nucleotides and inverted-dT nucleotides were synthesized in 1 micromole scale, purified by HPLC and verified by MALDI-ToF MS analysis.

TABLE 6
Sequence details of AP10-01 and AP10-01-81 aptamer with LNA-nucleotides
and an inverted-dT nucleotide

| SEQ ID | | |
|---|---|---|
| NO | Seq Name | Aptamer sequence |
| 6 | AP10- | ACAGTGTGGGCGGGCGGGTGGTTTGGAGCTGGTAGCGGTA-Inv-dT |
| | 01-L | |
| 9 | AP10- | GGACAGGACCACACCCAGCGACAGTGTGGGCGGGCGGGTGGTTTGGAG |
| | 01-81-L | CTGGTAGCGGTAGGCTCCTGTGTGTCGCTTTGT-Inv-dT |

### Example 3

The first approach was to pursue the TGpr1 D-dimer as a target

Future experiments were carried out on actual blood clots, manufactured as a target for each SELEX experiment.

### Blood Clot (Cotton Wool) Binding Analysis

### Blood Clot Target Preparation:

(1). Blood was anticoagulated with 0.4% sodium citrate and centrifuge at 2500 g to collect plasma. Spin again to remove remaining blood cells and froze at −80 degree as plasma stock.
(2). Add 100 ul of plasma to each wells of a 96-well plate.
(3). Add 10 ul of 220 mM CaCl₂) to each well (working concentration of 20 mM) to result in clot.
(4). Washing clots carefully with PBS with 1000 g for 5 min. Repeat 3 times.

### Testing Binding Capacity of Aptamers to Blood Clot

### A. Imaging Assay

(1) Blocking the blood clots with 100 ug/ml tRNA at RT for 1 h.
(2) After washing at 1000 g for 5 min. Adding 500 nM TGpr-1 aptamer and negative control aptamer (Library RNV75) to plate wells containing blood clots and incubate at RT for 1 h. Two wells per group.
(3) After incubation, washing clots with PBS with 1000 g for 5 min. Repeat 3 times.
(4) Add 100 ul of PBS to each well and forward to imaging using fluorescent microscope to compare the relative fluorescence density of TGpr-1 aptamer and control aptamers.

### B. ELONA Assay

(1) Blocking the blood clots with 100 ug/ml tRNA at RT for 1 h.
(2) After washing at 1000 g for 5 min. Adding 500 nM biotin conjugated TGpr-1 aptamer and control aptamer to plate wells containing blood clots and incubate at RT for 1 h. Two wells per group.
(3) After incubation, washing clots with PBS with 1000 g for 5 min. Repeat 3 times.
(4) Incubate clots with anti-biotin antibody (1:1000 dilution) at RT for 1 h.
(5) After incubation, washing clots with PBS with 1000 g for 5 min. Repeat 3 times.
(6) Add 100 ul of Quantablu HRP substrate and forward to Plate reader.

### C. Binding Affinity Assay

Same procedure with 2 but using a series of aptamer concentrations.

### Preparation of Insoluble Cross-Linked Fibrin (Cotton Wool Clot).

It was observed clear blood clots could be obtained by 1 h incubation of citrate treated plasma with 20 mM CaCl₂). After the time point of 1 h, the blood clots do not increase with time. Therefore, 1 h incubation was used for subsequent quantitative assay and Blood clot-SELEX to produce aptamers directly using Blood clot.

### Developing a Real-Time PCR Based Method for Binding Affinity Assay and Monitoring the Progress of Blood Clot-SELEX.

The insoluble nature of blood clots makes it difficult to measure the binding affinity of aptamers to them by plate reader based ELONA assay. To solve this problem, a Real-time PCR based quantitative method was developed for binding affinity assay and for monitoring the progress of Blood clot-SELEX. As Real-time PCR can accurately measure the amount of aptamers on a certain amount of blood clot after co-incubation and thorough washing, the quantitative data collected by this method can be used to plot a binding affinity curve as ELONA assay does (ELONA assay is a method to indirectly estimate the amount of aptamer via measuring the fluorescence signal of aptamers).

However, as this method need to use primer binding sites to enable PCR, only full-length aptamers can be directly measured. To measure truncated aptamers, a Taqman PCR-like method (similar with miRNA quantification) needed to be developed to introduce stem-loop structure to the ends of aptamers.

### Binding Capacity of Full Length AP10-01 Aptamer to Blood Clots

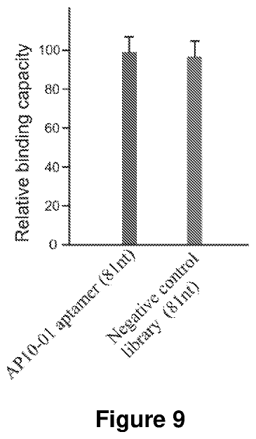

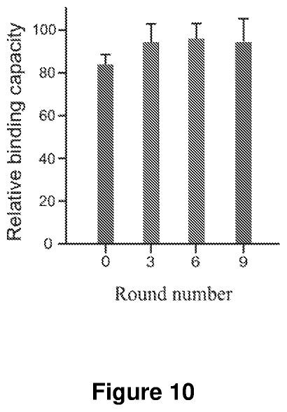

As demonstrated by FIG. 9, no specific binding capacity of AP10-01 aptamer to blood clot was observed. One of the reason is probably because, unlike the PBS conditions used for AP10-01 aptamer development, the binding capacity was performed in 75% plasma to mimic the in vivo environment. No prominent binding capacity was observed by qPCR quantification at Round 3, 6 and 9 (FIG. 10). To overcome this, SELEX will be performed for additional 5-10 rounds to enrich potential binders in a 75% plasma environment.

### Example 4

During Example 3, it was found that although the AP10-01 aptamer displays specific binding to the D-dimer, it does not show specific binding to the insoluble cross-linked fibrin (cotton wool clot). This is probably because the free D-dimer displays different conformational structure to the tested blood clot and the insoluble cross-linked fibrin cannot be recognised by aptamers developed using D-dimer.

It was further found that developing aptamers in PBS incubation buffer did not necessarily produce aptamers able to function effectively in plasma. Further experiments were carried out using plasma in the incubation buffer.

### Experiment Design of the Blood Clot-SELEX

Firstly, SELEX was conducted to directly target blood clot rather than free D-dimer protein, to maximise the probability of developing aptamers targeting to natural state blood clot. Secondly, rather than using the commonly used PBS incubation buffer, SELEX was performed in 75% plasma as incubation buffer. The purpose is to mimic the in vivo condition, to make sure the developed aptamers can be used in further clinical translation. Apart from mimicking in vivo conditions by using 75% plasma as incubation medium, the protein components of plasma (such as fibrinogen, fibrinopeptides) can be used directly as negative control. This is a highlight of this SELEX design, which ensures the developed aptamers can distinguish the differences between plasma proteins and the blood clot, and therefore enhance the binding specificity of the identified aptamers to blood clot.

### The Proper Creation of Blood Clot was Confirmed by Factor XIII Assay

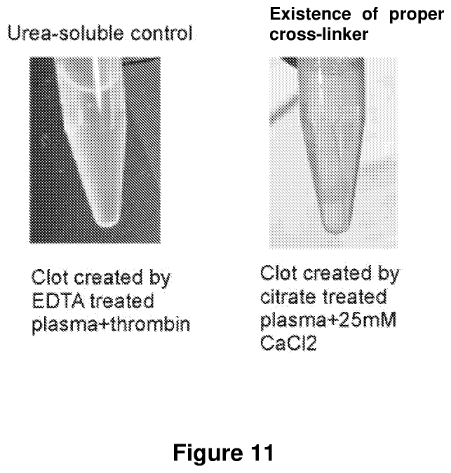

Providing proper blood clot target is important for aptamer development. To demonstrate the creation of proper crosslinks, Factor XIII assay was performed, by employing unstable blood clot control created via incubating EDTA-treated plasma and thrombin (20 units/mL). As shown in FIG. 11, compared with the blood clots prepared by incubating EDTA treated plasma and thrombin, the blood clot used in this project display stable cross-linkages. After 12 h incubation with 10 M Urea, while the control clot dissolved completely, the blood clot prepared by co-incubation of citrate treated plasma and Ca²⁺ did not display any noticeable change.

### The Initial SELEX Library can be Used for Blood Clot-SELEX in 75% Plasma

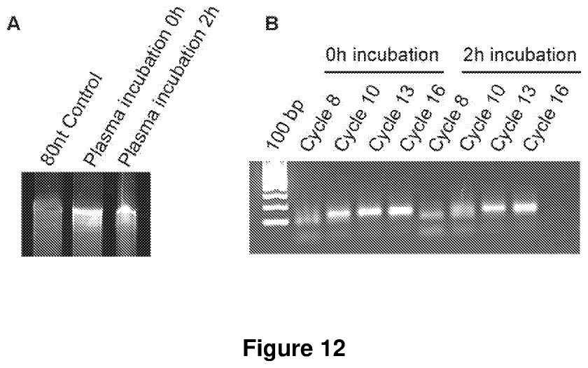

To perform SELEX directly in high-concentration of plasma, it is important for the synthesised initial single stranded DNA (ssDNA) library to be stable in the 75% plasma condition for at least one hour (the time for blood clot and ssDNA library co-incubation). Fortunately, as demonstrated by the denaturing gel analysis and PCR assay (FIG. 12), the initial library used is stable in 75% plasma for at least 2 hours. As shown in FIG. 12A, after 2 h incubation with the 75% plasma, no significant degradation of the ssDNA library was observed, and the ssDNA can be amplified by PCR with a similar efficacy to the untreated ssDNA control (FIG. 12B). As a result, this ssDNA library (RNV31) was used for the next Blood clot-SELEX.

Blood Clot-SELEX was Exterminated at Round 15 as Determined by qPCR-Based Sequence Enrichment Assessment

To increase the specificity of the developed aptamers, the selection conditions were adjusted with the progress of SELEX. Firstly, by gradually increasing the blocking stringency by adding yeast tRNA from 5 μg/mL for the first-round selection to 50 μg/mL for the late stage selection, to minimise the enrichment of non-specific binding. Secondly, by gradually reducing the amount of blood clot and increasing the amount of sub-library, to selectively identify sequences with higher binding capacity. To be specific, blood clot derived from 250 μL plasma was used for the first round and at the late stage of SELEX, 50 μL plasma was used for blood clot preparation, and the input libraries were gradually increased from 100 pmol for Round 2-5 to 300 pmol for Round 10 and afterwards.

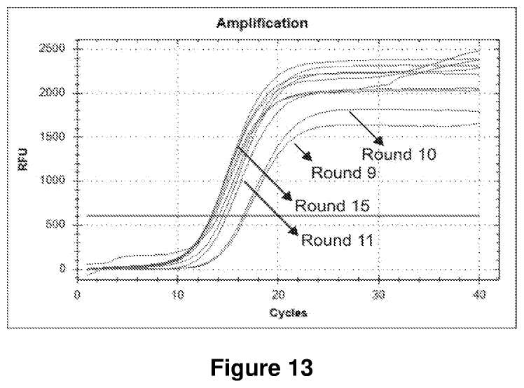

The progress of aptamer selection was monitored by a qPCR method as described in the Examples above. As expected, sequences showing binding to blood clot were enriched with the progress of SELEX. As demonstrated in FIG. 13, the enrichment reaches a plateau after Round 11, with Round 15 showing similar enrichment with Round 12, 13 and 14.

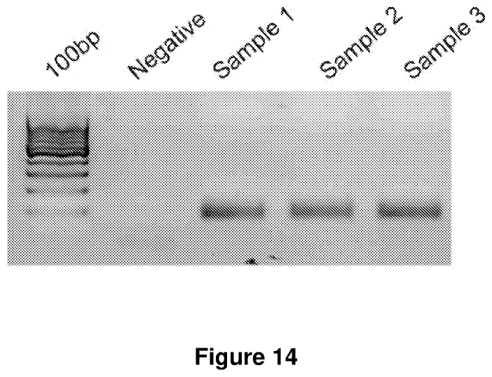

Consequently, the SELEX was stopped and sub-libraries from Round 3, Round 9 and Round 15 were amplified, purified (FIG. 14) and forwarded for next generation sequencing and bioinformatics analysis.

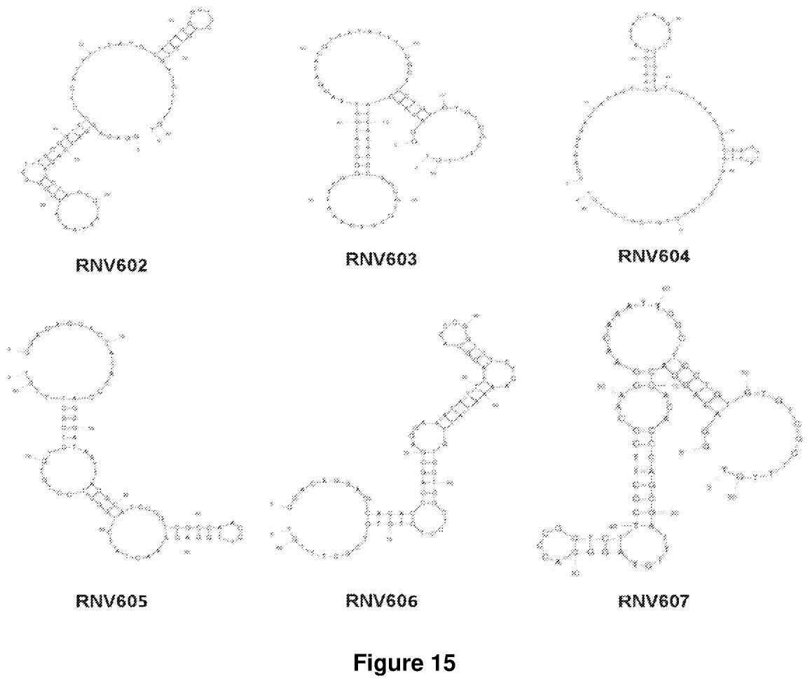

After next generation sequencing, six sequence candidates displaying higher proportion in abundance and structural complexity (Table 7) were selected based on bioinformatics analysis and tertiary structure prediction (FIG. 15).

TABLE 7
Sequence details of six further aptamers directed against fibrin

| SEQ | Seq | |
|---|---|---|
| ID NO | Name | Aptamer sequence (5′-3′) |
| 10 | RNV602 | GGACAGGACCACACCCAGCGTAATAATATGGGGCTTGCGGTCCCCGATTTTTAT |
| | | GCACACGGCTCCTGTGTGTCGCTTTGT |
| 11 | RNV603 | GGACAGGACCACACCCAGCGTGCGTGAAATTCGGGGCATGGTTAGGATAGGTA |
| | | ATATTTTGGCTCCTGTGTGTCGCTTTGT3 |
| 12 | RNV604 | GGACAGGACCACACCCAGCGGATACCTGAAGCTGGCCCACATTTATCTACGCGA |
| | | TATATTGGCTCCTGTGTGTCGCTTTGT |
| 13 | RNV605 | GGACAGGACCACACCCAGCGATAATTAGGCATCCGGTCGCCAACCTTGGAG |
| | | AAACTATCTGGCTCCTGTGTGTCGCTTTGT |
| 14 | RNV606 | GGACAGGACCACACCCAGCGGATAACTAGGCACCCGGTGTCGTTATTATGACAA |
| | | GATATTGGCTCCTGTGTGTCGCTTTGT |
| 15 | RNV607 | GGACAGGACCACACCCAGCGAGGAAATATTTAGGCACCCGGTCTCTGAAAATAT |
| | | TGTGCTGGCTCCTGTGTGTCGCTTTGT |

### Binding Capacity of the Identified Aptamer Sequences

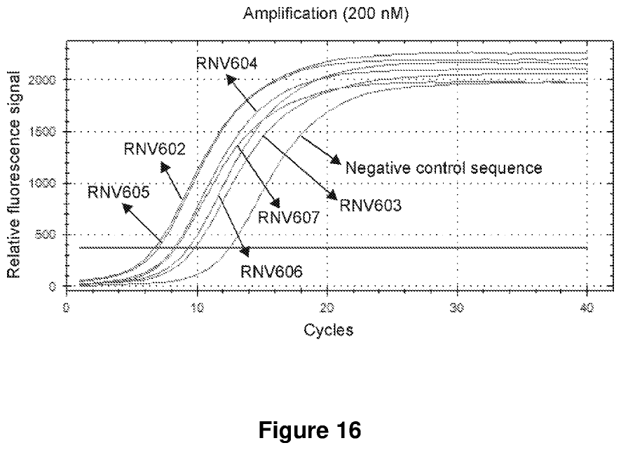

The binding capacity of the six aptamer candidates was tested by qPCR method. As shown in FIG. 16, compared with the negative control sequence (an irrelevant aptamer having identical primer binding site with RNV 602-607 sequences), all of the selected sequences can target blood clot, with sequence RNV602 and RNV605 displaying the strongest binding to the blood clot.

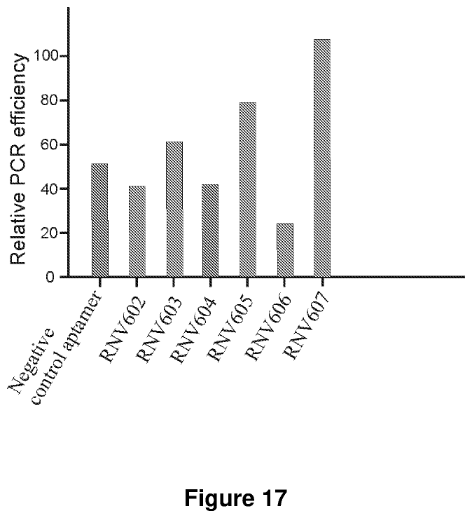

However, it should be noticed that aptamer sequences sharing the identical primer binding site may have different PCR efficiencies due to PCR bias. To eliminate the potential effect of PCR bias on the binding capacity as measured by qPCR-based binding capacity assay, a separate PCR efficiency analysis was performed to study the relative PCR efficiency of these aptamer candidate sequences (FIG. 17).

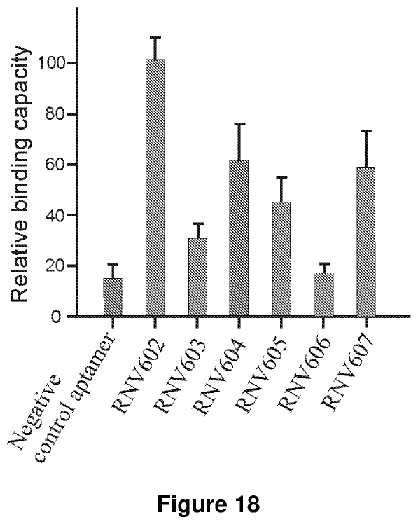

The normalised binding capacity, after eliminating the effect of PCR bias, is shown in FIG. 18. As can be seen, RNV602 displays a strong binding capacity to blood clot, with a dissociation constant (K_{d}) of 46.15 nM to the blood clot.

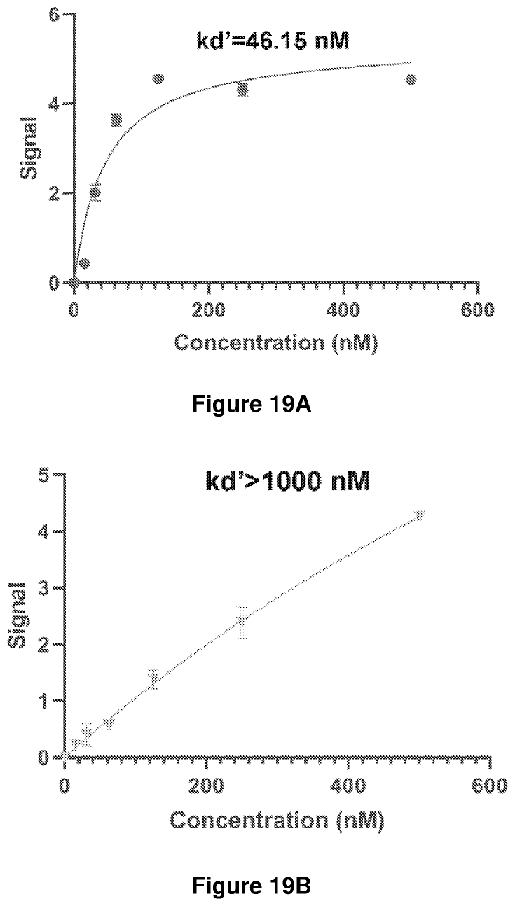

The binding affinity of this sequence was then studied by co-incubating the blood clot target with a series of aptamer concentrations. According to analysis, the binding affinity of RNV602 is 46.15 nM to blood clot (FIG. 19A). To assess the binding specificity of RNV602 aptamer, it was incubated with plasma proteins immobilised on a 96 well plate using amine-binding plate. As demonstrated in FIG. 19B, this aptamer does not show binding to proteins derived from plasma, suggesting this aptamer is likely to target blood clot in blood vessels while not targeting non-specifically binding plasma proteins.

### Example 5

### Further Testing Identified RNV602 and RNV605 Aptamers

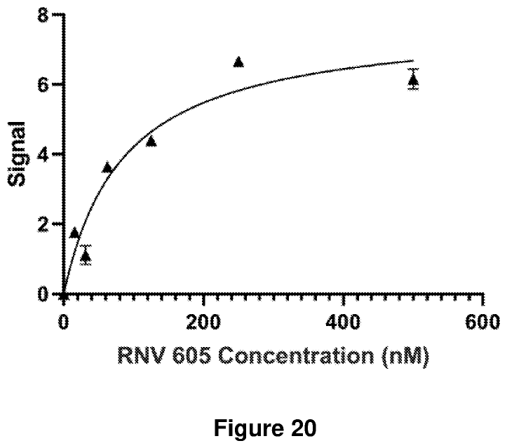

Further qPCR based binding analysis was conducted to compare the binding capacity of RNV603, RNV604, RNV605, RNV606 and RNV607 aptamers. As demonstrated in FIG. 20, another aptamer sequence, RNV605, displaying a binding affinity of 85.81 nM, was also identified.

### Synthesis of FAM-Conjugated RNV602 and RNV605 Sequences for Further Binding Property Analysis

Although the qPCR based binding assay developed for this project provide an opportunity to estimate the binding property of aptamers to solid blood clot, the amplification bias with such PCR-based quantitative method could potentially compromise its accuracy. To provide more reliable and accurate binding information, fluorescent dye (FAM)-labelled RNV602 and RNV605 aptamers were synthesised for further validations, as shown in Table 8.

TABLE 8
Sequences of the FAM-conjugated RNV602 and RNV605 aptamers

| SEQ | | | |
|---|---|---|---|
| ID NO | Identifier | Length | DNA sequence |
| 10 | FAM-RNV602 | 81 | 5′-FAM- |
| | | | GGACAGGACCACACCCAGCGTAATAATATGGGGCTTGCGGT |
| | | | CCCCGATTTTTATGCACACGGCTCCTGTGTGTCGCTTTGT-3′ |
| 13 | FAM-RNV605 | 81 | 5′-FAM- |
| | | | GGACAGGACCACACCCAGCGATAATTAGGCATCCGGTCGCC |
| | | | AACCTTGGAGAAACTATCTGGCTCCTGTGTGTCGCTTTGT-3′ |

### RNV602 and RNV605 Aptamers Display Prominent Binding to Blood Clots Under Fluorescence Microscopy

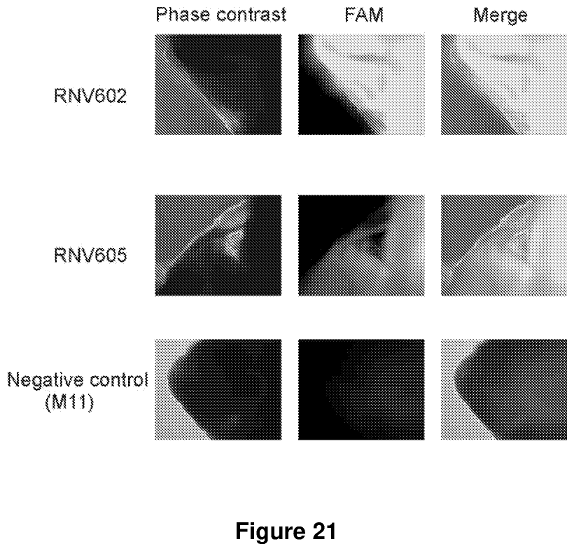

To qualitatively test the binding of both RNV602 and RNV605 to the blood clots, fluorescence microscopy assay was performed, using an irreverent M11 aptamer as a negative control. The M11 aptamer has as an identical molecular weight and FAM-tag to RNV602 and RNV605). Compared with the negative control M11 aptamer, both RNV602 and RNV605 demonstrated significant binding to blood clots prepared overnight using plasma and 25 mM Ca²⁺ incubation (FIG. 21).

### The Binding of RNV602 and RNV605 to Blood Clots is Specific

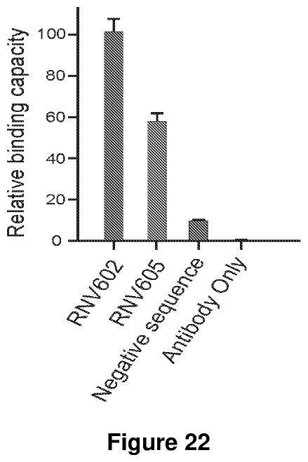

Quantitative studies were conducted to evaluate the binding of RNV602 and RNV605 aptamers to blood clots via the ELONA assay as explained previously. As shown in FIG. 22, overnight blood clots were incubated with 200 nM of the indicated aptamer for 0.5 h, followed by thorough washing. While the negative control M11 aptamer does not display binding to blood clot, both RNV602 and RNV605 aptamers showed strong binding with the tested 200 nM concentration, with a 10-fold and 6-fold higher binding capacity than the untreated antibody-only group.

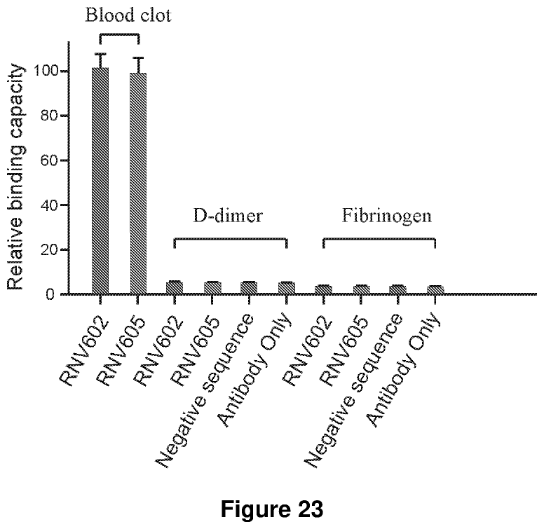

Apart from employing the negative control M11 aptamer to control the possibility of the non-specific binding of oligonucleotides themselves to the blood clot, D-dimer and fibrinogen proteins were also used to check the specificity of the identified RNV602 and RNV605 aptamers. As demonstrated in FIG. 23, both RNV602 and RNV605 did not display significant binding to D-dimer and fibrinogen proteins under the tested 100 nM concentration of aptamers. Therefore, the binding of RNV602 and RNV605 aptamers to blood clots is specific to the clots, and the aptamers do not bind either fibrinogen precursor or D-dimer degradation proteins.

### RNV602 and RNV605 Display Picomolar Level Binding Affinity to Blood Clots

After testing the binding specificity of RNV602 and RNV605 aptamers to blood clots, the binding affinity assay was repeated by using the established ELONA assay. Previously, the qPCR assay was developed to evaluate binding affinity of blood clot aptamers considering the physical state (solid) of the target which was difficult to immobilise on any solid surface. However, as observed during tests, the PCR amplification efficacies were different for each aptamers which could negatively affect the accuracy of this method. To circumvent this issue, the effectiveness of the traditional ELONA with some modifications was tested by using the FAM-conjugated aptamer and anti-FAM antibody.

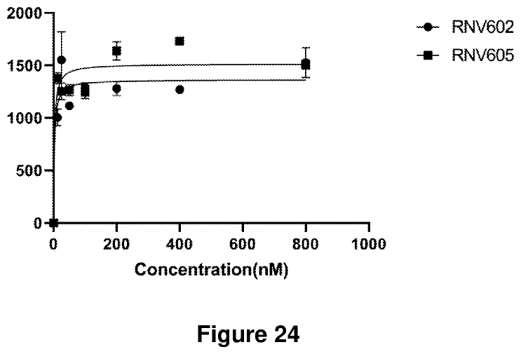

Based on the approximately 50-80 nM binding affinity as measure by qPCR methods, the binding affinity was first tested with a concentration range from 12.5 nM to 800 nM as shown in FIG. 24. Surprisingly, both aptamers demonstrated strong binding capacity in this test, with the lowest concentration of 12.5 nM immediately reached the measurement plateau.

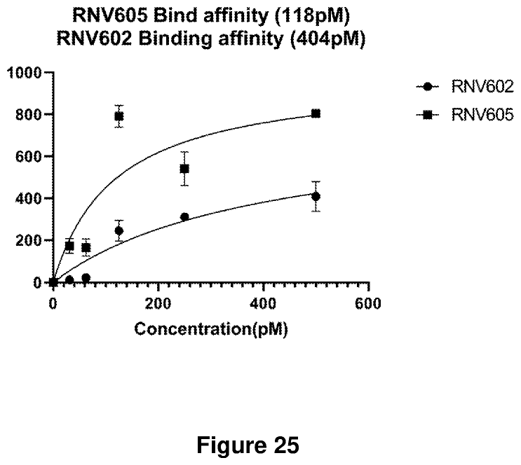

Consequently, the concentration for both aptamers was lowered to a range from 30 pM to 500 pM, in order to see a progressive increase in the signal with the increase of aptamer concentration. A more reliable binding affinity value of 118 pM and 404 pM for aptamer RNV605 and RNV602 respectively was detected (FIG. 25).

From the above tests, two strong binding aptamers, RNV602 and RNV605 were identified, as confirmed by the imaging assay and fluorescence-based ELONA assay. These aptamers not only demonstrated strong binding affinity to the overnight blood clots, but also showed sufficient specificity.

### Example 6

### Confirm the Binding Specificity of RNV602 and RNV605 Aptamers to Blood Clots Via Mouse Plasma

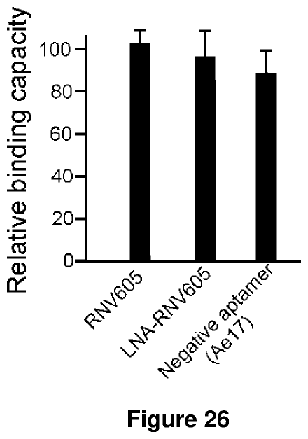

Although both RNV602 and RNV605 did not demonstrate binding capacity to D-dimer and fibrinogen proteins, such control approach cannot exclude the potential non-specific binding of aptamers to the cross-link structure of blood clots. To best mimic the cross-link structure, collagen protein (with cross-link structure) was employed. As demonstrated in FIG. 26, neither RNV602 nor RNV605 displayed specific binding to collagen protein.

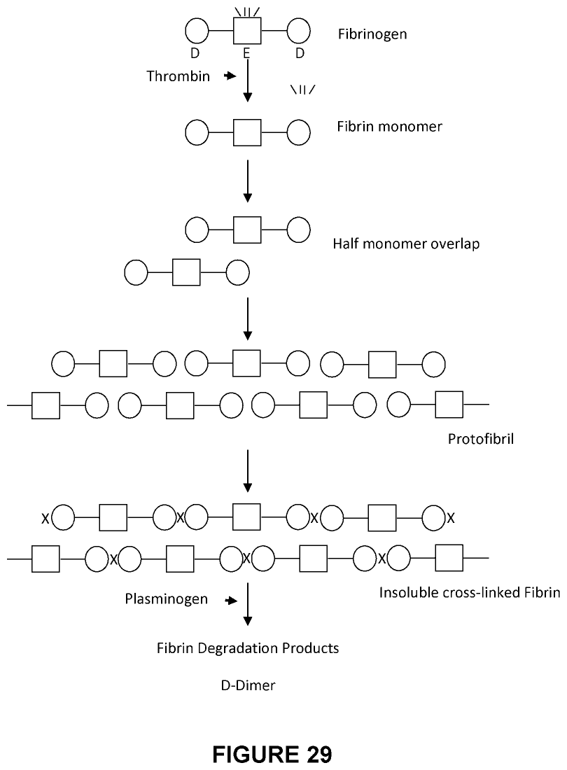

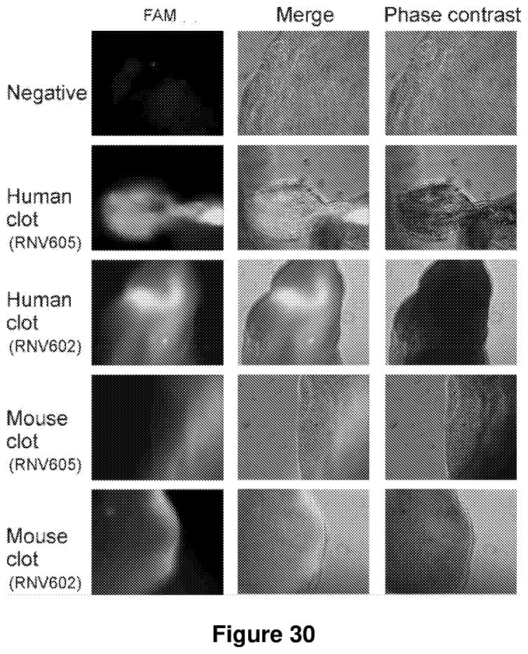

However, although collagen possesses a blood clot-like cross-link structure, it still represents an imperfect negative control, as the selected aptamers could potentially bind non-specifically to other objects in the blood vessel. To provide further result, blood clots prepared from mouse plasma were tested. As shown in FIG. 29, both RNV602 and RNV605 showed binding to mouse derived clots.

### Example 7

### Further Modification of RNV605 by LNA Technology

To overcome any stability issues associated with an unmodified ssDNA aptamer RNV605 was modified via LNA technology, as demonstrated in Table 9.

TABLE 9
Sequences of the FAM-conjugated RNV605 and LNA-RNV605 aptamers

| SEQ | | | |
|---|---|---|---|
| ID NO | Identifier | Length | DNA sequence |
| 13 | FAM- | 81 | 5′-FAM- |
| | RNV605 | | GGACAGGACCACACCCAGCGATAATTAGGCATCCGGTCGCCAAC |
| | | | CTTGGAGAAACTATCTGGCTCCTGTGTGTCGCTTTGT-3′ |
| 13 | FAM-LNA- | 81 | 5′-FAM- |
| | RNV605 | | GGACAGGACCACACCCAGCGATAATTAGGCATCCGGTCG [symbol] CAAC |
| | | | CTTGGAGAAACTATCTGG [symbol] TCCTGTGTG [symbol] CG [symbol] TTTGT-InvdT-3′ |

### LNA-RNV605 Aptamer is Stable in 90% Serum

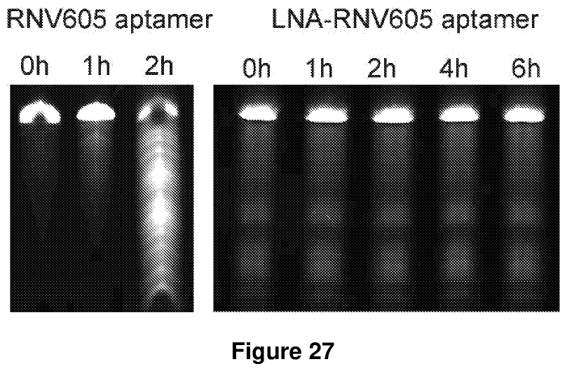

LNA modification greatly improved the stability of RNV605. While the unmodified RNV605 start to degrade 1 h after 90% serum incubation and displayed dramatic degradation at the time point of 2 h, the LNA-RNV605 sequence did not show prominent degradation even 6 h after 90% serum incubation (FIG. 27).

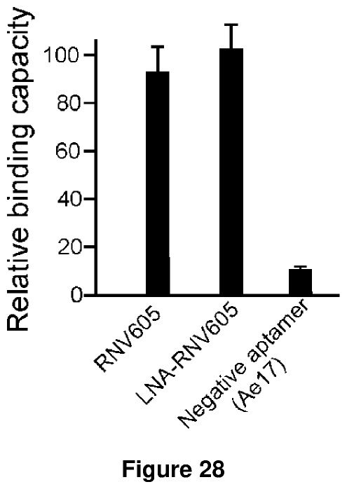

Importantly, the LNA modification did not affect the binding capacity of RNV605 to blood clots. As shown in FIG. 28, both RNV605 and LNA-RNV605 displayed 8-10 times higher binding capacity than the negative control AE17 sequence, comparable with previous results. No significant difference between RNA605 and LNA-RNV605 was observed in three independent tests. These results suggest that LNA-RNV605 possesses sufficient serum stability and binding capacity to blood clots.

From the above tests, the aptamer LNA-RNV605 displayed improved serum stability but the same binding capacity as the original RNV605.

### Example 8

### Summary of Aptamers Developed

TABLE 10
Aptamers developed and tested against fibrin

| SEQ ID NO | Identifier | DNA sequence |
|---|---|---|
| 1 | EM1 | 5′ Biotin- |
| | | ACAAAGCGACACACAGGAGCCCAATGATGCTGTTATGGCGCTTTG |
| | | TGGACAGGACCACACCCAGCG-3′ |
| 2 | AP1 | 5′ Biotin- |
| | | ACAAAGCGACACACAGGAGCCCGGTCGGCGGGGGGGCGGGTGG |
| | | AGAACGAGGTAGGGGTCAGGACAGGACCACA000AGCG-3′ |
| 3 | AP3 | 5′ Biotin- |
| | | ACAAAGCGACACACAGGAGCCCGGTCGGCGGGTGGGCGGGTGG |
| | | AGAACGAGGTAGGGGTCAGGACAGGACCACACCCAGCG-3′ |
| 4 | EM9-02 | AAGGGAAAGTGTATACACTATGGCT |
| 5 | MB8-01 | CGGTCGGCGGGGGGGCGGGTGGAGAACGAGGTAGGGGTCA |
| 6 | AP10-01 | ACAGTGTGGGCGGGCGGGTGGTTTGGAGCTGGTAGCGGTA |
| 7 | AP10-06 | TCATCGTGGGTGGGTTGGGTGGAACGACGTAGAGGGGGTG |
| 8 | AP10-07 | CGTTCGGCGGGGGGGCGGGTGGAGAACGAGGTAGGGGTCA |
| 9 | AP-10-01- | GGACAGGACCACACCCAGCGACAGTGTGGGCGGGCGGGTGGTTT |
| | 81 | GGAGCTGGTAGCGGTAGGCTCCTGTGTGTCGCTTTGT |
| 10 | RNV602 | GGACAGGACCACACCCAGCGTAATAATATGGGGCTTGCGGTCCC |
| | | CGATTTTTATGCACACGGCTCCTGTGTGTCGCTTTGT |
| 11 | RNV603 | GGACAGGACCACACCCAGCGTGCGTGAAATTCGGGGCATGGTTA |
| | | GGATAGGTAATATTTTGGCTCCTGTGTGTCGCTTTGT |
| 12 | RNV604 | GGACAGGACCACACCCAGCGGATACCTGAAGCTGGCCCACATTTA |
| | | TCTACGCGATATATTGGCTCCTGTGTGTCGCTTTGT |
| 13 | RNV605 | GGACAGGACCACACCCAGCGATAATTAGGCATCCGGTCGCCAAC |
| | | CTTGGAGAAACTATCTGGCTCCTGTGTGTCGCTTTGT |
| 14 | RNV606 | GGACAGGACCACACCCAGCGGATAACTAGGCACCCGGTGTCGTT |
| | | ATTATGACAAGATATTGGCTCCTGTGTGTCGCTTTGT |
| 15 | RNV607 | GGACAGGACCACACCCAGCGAGGAAATATTTAGGCACCCGGTCTC |
| | | TGAAAATATTGTGCTGGCTCCTGTGTGTCGCTTTGT |

### Example 9

### Alternative Targeting Motifs

As previously reported (Sci Rep. 2013; 3: 2604, doi: 10.1038/srep02604), the peptide fragment 201-216 (fibrinopeptide; Cys-Asn-Ile-Pro-Val-Val-Ser-Gly-Lys-Glu-Cys-Glu-Glu-Ile-Ile-Arg) is in the Bβ chain of fibrinogen, which has been discovered as an uncovered region that develops in the fibrin clot during its formation, and therefore represents a promising target motif for developing high-affinity aptamers targeting fibrin clots.

To explore this possibility, a nickel-plate based Peptide-SELEX was performed using 6× Histidine-tagged 201-216 peptide segment of fibrinogen. However, although preparing solid protein phase using a nickel coated plate (Histidine binding plate) ensures efficient SELEX procedure by allowing easy handling, stringent washing and quantitative adjustment of the protein amount for individual rounds, the efficiency of this protein immobilisation method could be affected by two issues. Firstly, the natural structural conformation of the fibrinogen peptide could undergo significant change during immobilisation, which ultimately affects the applicability of the developed aptamer to native fibrinopeptide. Secondly, during SELEX, the library sequences might bind to the plastic matrices and causes non-specific selection.

To facilitate a high-efficient SELEX and the identification of successful aptamers capable of recognising the native state fibrinopeptide, a competitive elution strategy (an approach to elute highly specific aptamers using excess amount of target solution from the surface-immobilised target after an initial DNA library incubation step) was introduced. After target incubation and extensive washes, 100 pmol free fibrinopeptide (more than 10 times of the immobilised protein bait) was added to the immobilised fibrinogen peptide/ssDNA library mixture to elute ssDNA sequences which are able to recognise the native state free fibrinopeptide. In addition, to improve the specificity of the selected aptamers, a negative selection using His-tagged Spike protein immobilized Ni-NTA well was performed for each round. Firstly, the ssDNA library from individual rounds firstly incubated with the spike protein immobilized well for 1 h to remove the nonspecific binding sequences, followed by transferring the supernatant to the positive selection well (immobilized with His tagged fibrinopeptide).

The SELEX was performed consecutively for 10 rounds. Sub-libraries of round 3, round 6, round 10 were submitted for next generation sequencing. After next generation sequencing, six sequence candidates displaying higher proportion in abundance and structural complexity (Table 11) were selected based on bioinformatics analysis and tertiary structure prediction.

TABLE 11
Sequence details of six aptamers directed against uncover region of fibrin

| SEQ ID NO | Identifier | DNA sequence |
|---|---|---|
| 16 | RNV744 | 5′ biotin- |
| | | GGACAGGACCACACCCAGCGTGGTGGTGAAGAGGTGGGAGGGAGG |
| | | GCCCCGCGATGGTAAGGCTCCTGTGTGTCGCTTTGT 3′ |
| 17 | RNV745 | 5′ biotin- |
| | | GGACAGGACCACACCCAGCGGGCGGGCGGGGGAGGTGGAGGAAAT |
| | | GTGGGGAATGGGTGCGGCTCCTGTGTGTCGCTTTGT 3′ |
| 18 | RNV746 | 5′ biotin- |
| | | GGACAGGACCACACCCAGCGGTGGGGGGGGAGGAGGGTGGGGAG |
| | | AAGGGTCAATGGTGGTGGCTCCTGTGTGTCGCTTTGT 3′ |
| 19 | RNV747 | 5′ biotin- |
| | | GGACAGGACCACACCCAGCGGGCGGGCGGGGGAGGTGGAGGAAAT |
| | | GTGGGGAATGGGTACGGCTCCTGTGTGTCGCTTTGT 3′ |
| 20 | RNV748 | 5′ biotin- |
| | | GGACAGGACCACACCCAGCGGTGGGGGGGGAGGAGGGTGGGGAT |
| | | AAGGGTCAATGGTGGTGGCTCCTGTGTGTCGCTTTGT 3′ |
| 21 | RNV749 | 5′ biotin- |
| | | GGACAGGACCACACCCAGCGCGGTAAGTGGTAGGGGAGTGGCGGG |
| | | ATTGGCTTAGGGTCTGGCTCCTGTGTGTCGCTTTGT 3′ |

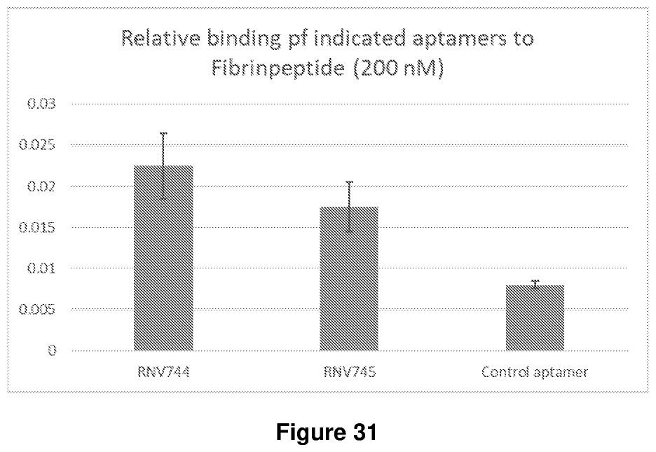

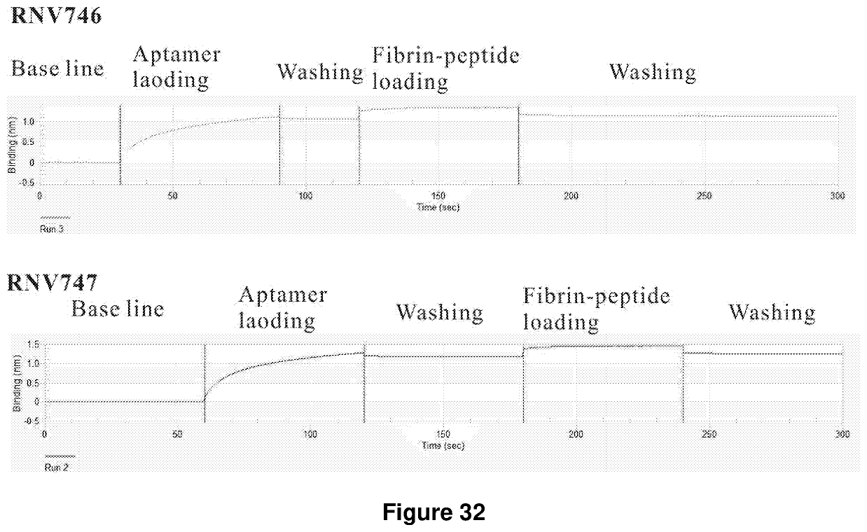

The binding capacity of the six aptamer candidates were tested by either ELONA assay or surface plasmon resonance assay. As shown in FIG. 31, according to the ELONA assay aptamers RNV744 and RNV745 displayed enhanced binding to the fibrinopeptide compared to the negative control aptamer. According to the biolayer interferometry assay (FIG. 32), aptamers RNV746 and RNV747 also showed fibrinopeptide binding.

1. An isolated or purified aptamer adapted to bind fibrin.

2. The aptamer of claim 1 wherein the aptamer does not bind:
i) one or more of the following: fibrinogen, fibrin monomer, non-cross-linked fibrin, protofibril, soluble fibrin, fibrin degradation product, D-dimer or other protein components of plasma; or
ii) any of the following: fibrinogen, fibrin monomer, non-cross-linked fibrin, protofibril, soluble fibrin, fibrin degradation product, D-dimer or other protein components of plasma.

3. The aptamer of claim 1 wherein the aptamer is chosen from:
i) the sequences provided in Tables 10 and 11;
ii) any of SEQ ID Nos: 1-21;
iii) SEQ ID NO: 10 or 13;
iv) SEQ ID NO: 16 or 17;
v) sequences which have at least 85% sequence similarity to any one of (i) to (iv); and/or
vi) sequences which have at least 85% sequence identity to any one of (i) to (iv).

4. The aptamer of claim 1 wherein the aptamer is labelled with a detection means.

5. The aptamer of claim 4 wherein the detection means is one or more of the following: radiotracers, fluorescent dyes, drug molecules, electrochemical signalling molecules, magnetic and polymeric nanoparticles, lipids and liposomes, magnetic labels, iodine X-ray blocking compounds, radiotracers.

6. The aptamer of claim 1 wherein the aptamer has a binding affinity for fibrin of between 30 pM and 500 pM when tested by qPCR-based binding capacity assay.

7. The aptamer of claim 1 wherein the aptamer is modified by the inclusion of one or more of the following modifications: LNA-nucleotides, 2′-Fluoro nucleotides, 2′-O-Methyl nucleotides, 2′-OMOE nucleotides, PMO, unlocked nucleic acid nucleotides, L-DNA/L-RNA nucleotides and inverted-dT nucleotides.

8. A method for the detection of fibrin in a subject, said method comprising the steps of:
a) administering to the plasma of a subject a purified and isolated aptamer adapted to bind fibrin;
b) detecting the aptamer bound to the fibrin.

9. (canceled)

10. A pharmaceutical composition for the detection of fibrin in a subject, the composition comprising:
a) a purified and isolated aptamer according to claim 1; and
b) one or more pharmaceutically acceptable carriers and/or diluents.

11. A kit for the detection of fibrin in a subject, said kit comprising:
a) a purified and isolated aptamer adapted to bind fibrin; and
b) instructions for use.

12. A method to produce aptamers that target fibrin, said method comprising:
a) performing SELEX using D-dimer as the positive target protein and a single fibrinogen D domain as the subtraction target protein;
b) performing SELEX using freshly produced blood clot as the positive target protein and fibrinogen, a single fibrinogen D domain, D-dimer, other normal plasma components as the subtraction target proteins; or
c) performing SELEX using (i) peptide 201-216 in fibrinogen as the target protein.

13. The method of claim 12 wherein the method is carried out with: no plasma; plasma from people who do not have clot in their circulation; or plasma from patients who have clot abnormally present in their circulation, in the SELEX medium.

14. A method to produce aptamers that target fibrin, said method comprising performing SELEX using (i) clot as the positive target protein and (ii) plasma from people who do not have clot in their circulation in the SELEX medium.

15. A method to produce aptamers that target fibrin, said method comprising performing SELEX using (i) clot as the positive target protein and (ii) plasma from patients who have clot abnormally present in their circulation in the SELEX medium.

16. The method of claim 14, wherein the plasma is obtained from a different subject for each round of SELEX, and wherein the last round of SELEX uses plasma pooled from all of the previous rounds of SELEX.

17. The method of claim 15, wherein the plasma is obtained from a different subject for each round of SELEX, and wherein the last round of SELEX uses plasma pooled from all of the previous rounds of SELEX.


SEQUENCE LISTING (15 sequences)

SEQ ID NO: 1 - DNA, 66 bp, Homo sapiens
acaaagcgacacacaggagcccaatgatgctgttatggcgctttgtggacaggaccacacccagcg

SEQ ID NO: 2 - DNA, 81 bp, Homo sapiens
acaaagcgacacacaggagcccggtcggcgggggggcgggtggagaacgaggtaggggtcaggacaggaccacacccagcg

SEQ ID NO: 3 - DNA, 81 bp, Homo sapiens
acaaagcgacacacaggagcccggtcggcgggtgggcgggtggagaacgaggtaggggtcaggacaggaccacacccagcg

SEQ ID NO: 4 - DNA, 25 bp, Homo sapiens
aagggaaagtgtatacactatggct

SEQ ID NO: 5 - DNA, 40 bp, Homo sapiens
cggtcggcgggggggcgggtggagaacgaggtaggggtca

SEQ ID NO: 6 - DNA, 40 bp, Homo sapiens
acagtgtgggcgggcgggtggtttggagctggtagcggta

SEQ ID NO: 7 - DNA, 40 bp, Homo sapiens
tcatcgtgggtgggttgggtggaacgacgtagagggggtg

SEQ ID NO: 8 - DNA, 40 bp, Homo sapiens
cgttcggcgggggggcgggtggagaacgaggtaggggtca

SEQ ID NO: 9 - DNA, 81 bp, Homo sapiens
ggacaggaccacacccagcgacagtgtgggcgggcgggtggtttggagctggtagcggtaggctcctgtgtgtcgctttgt

SEQ ID NO: 10 - DNA, 81 bp, Homo sapiens
ggacaggaccacacccagcgtaataatatggggcttgcggtccccgatttttatgcacacggctcctgtgtgtcgctttgt

SEQ ID NO: 11 - DNA, 81 bp, Homo sapiens
ggacaggaccacacccagcgtgcgtgaaattcggggcatggttaggataggtaatattttggctcctgtgtgtcgctttgt

SEQ ID NO: 12 - DNA, 81 bp, Homo sapiens
ggacaggaccacacccagcggatacctgaagctggcccacatttatctacgcgatatattggctcctgtgtgtcgctttgt

SEQ ID NO: 13 - DNA, 81 bp, Homo sapiens
ggacaggaccacacccagcgataattaggcatccggtcgccaaccttggagaaactatctggctcctgtgtgtcgctttgt

SEQ ID NO: 14 - DNA, 81 bp, Homo sapiens
ggacaggaccacacccagcggataactaggcacccggtgtcgttattatgacaagatattggctcctgtgtgtcgctttgt

SEQ ID NO: 15 - DNA, 81 bp, Homo sapiens
ggacaggaccacacccagcgaggaaatatttaggcacccggtctctgaaaatattgtgctggctcctgtgtgtcgctttgt